(19) United States
(12) Patent Application Publication
(10) Pub. No.: US 2025/0179464 A1
(43) Pub. Date: Jun. 5, 2025

(54) METHODS AND COMPOSITIONS FOR EDITING SBDS GENE

(71) Applicant: The Children’s Medical Center Corporation, Boston, MA (US)

(72) Inventor: Christian Brendel, Boston, MA (US)

(21) Appl. No.: 18/947,543

(22) Filed: Nov. 14, 2024

Related U.S. Application Data
(60) Provisional application No. 63/548,479, filed on Nov. 14, 2023.

(51) Int. Cl. C12N 9/80; A61P 19/08; C12N 9/22; C12N 15/11; C12N 15/90

(52) U.S. Cl. CPC C12N 9/80; A61P 19/08; C12N 9/22; C12N 15/11; C12N 15/907; C12Y 305/04001; C12N 2310/20

(57) ABSTRACT

Provided herein is a base editor system for editing the SBDS gene comprising a base editor protein and a guide polynucleotide, wherein the base editor protein comprises a polynucleotide programmable DNA binding domain and a cytosine deaminase domain. Methods, edited cells, and compositions thereof are also described herein.

### CROSS-REFERENCE TO RELATED APPLICATIONS

This application claims benefit of U.S. Provisional Application No. 63/548,479, filed Nov. 14, 2023, incorporated herein by reference in its entirety.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Nov. 13, 2024, is entitled “11624-011Wo_ST26.xml”, and is 16,279 bytes in size.

### BACKGROUND

About 90% of patients with Shwachman-Diamond-Syndrome (SDS) carry a mutation in intron 2 of the SBDS-gene (named 258+2 C to T), which leads to defective splicing and loss of protein expression. These patients suffer from multiple defects, but the life-threatening manifestation is bone marrow failure, i.e., the inability to produce sufficient numbers of blood cells. A high proportion of patients (30%) develops myelodysplastic syndrome and acute myeloid leukemia (AML). Currently the only curative treatment is bone marrow transplantation from a matched donor. Described herein are base editors and compositions thereof used for the treatment of SDS.

### SUMMARY

Described herein is the use of base editing technology to produce genetic modification of the SBDS gene. Accordingly, one aspect described herein provides a base editor system for editing the SBDS gene comprising a base editor protein and a guide polynucleotide, wherein the base editor protein comprises a polynucleotide programmable DNA binding domain and a cytosine deaminase domain.

In one embodiment of any aspect herein, the polynucleotide programmable DNA binding domain is a SpRY variant of a Streptococcus pyogenes Cas9 (SpCas9).

In one embodiment of any aspect herein, the cytosine deaminase domain is evoFERNY-SpRY.

In one embodiment of any aspect herein, the guide polynucleotide comprises a nucleic acid sequence of SEQ ID NO: 1.

In one embodiment of any aspect herein, the cytosine deaminase domain is an evoFERNY-SpRY and the guide polynucleotide comprises a nucleic acid sequence of SEQ ID NO: 1.

In one embodiment of any aspect herein, the cytosine deaminase domain is selected from the group consisting of evoCDA1-SpG; evoCDA1-SpRY; evoAPOBEC1-SpG; evoAPOBEC1-SpRY; evoFERNY-SpG; and evoRERNY-SpRY.

In one embodiment of any aspect herein, the guide polynucleotide is selected from a sequence selected from the group consisting of: a nucleic acid sequence of SEQ ID NO: 2-5.

In one embodiment of any aspect herein, the cytosine deaminase domain is an evoCDA1-SpG and the guide polynucleotide comprises a nucleic acid sequence selected from SEQ ID NO: 2-4.

In one embodiment of any aspect herein, the cytosine deaminase domain is an evoCDA1-SpRY and the guide polynucleotide comprises a nucleic acid sequence selected from SEQ ID NO: 2-4.

In one embodiment of any aspect herein, the cytosine deaminase domain is an evoAPOBEC1-SpG and the guide polynucleotide comprises a nucleic acid sequence of SEQ ID NO: 5.

In one embodiment of any aspect herein, the cytosine deaminase domain is an evoAPOBEC1-SpRY and the guide polynucleotide comprises a nucleic acid sequence of SEQ ID NO: 5.

In one embodiment of any aspect herein, the cytosine deaminase domain is an evoFERNY-SpG and the guide polynucleotide comprises a nucleic acid sequence of SEQ ID NO: 5.

In one embodiment of any aspect herein, the cytosine deaminase domain is an evoRERNY-SpRY and the guide polynucleotide comprises a nucleic acid sequence of SEQ ID NO: 5.

In one embodiment of any aspect herein, the SBDS gene comprises a T258C mutation.

In one embodiment of any aspect herein, the SBDS gene is edited at the nucleotide 258.

In one embodiment of any aspect herein, the base editor system corrects the T258C mutation.

In one embodiment of any aspect herein, correcting the T258C mutation results in at least one of: correct splicing, increased SBDS gene product expression, increased cell proliferation, increased ribosome maturation, and increased protein synthesis in the cell.

In one embodiment of any aspect herein, the cell is selected from the group consisting of: bone marrow cell a bone marrow cell, a somatic stem cell, a progenitor cell, a hematopoietic stem cell, or a hematopoietic progenitor cell.

Another aspect provided herein describes a base editor system comprising a base editor protein and a guide polynucleotide, wherein the base editor protein is evoFERNY-SpRY and the guide polynucleotide has sequence of SEQ ID NO: 1

Another aspect provided herein describes a method of editing a cell, the method comprising contacting a cell with any of the base editor systems described herein.

In one embodiment of any aspect herein, cell comprises a mutation in the SBDS gene. In one embodiment of any aspect herein, the mutation in the SBDS gene is a T258C mutation. In one embodiment of any aspect herein, the base editor corrects the T285C mutation.

In one embodiment of any aspect herein, the cell is isolated prior to contacting.

In one embodiment of any aspect herein, correcting the T258C mutation results in at least one of: correct splicing, increased SBDS gene product expression, increased cell proliferation, increased ribosome maturation, and increased protein synthesis in the cell.

In one embodiment of any aspect herein, the cell is obtained from a subject diagnosed as having Shwachman-Diamond-Syndrome (SDS) or at risk of having Shwachman-Diamond-Syndrome (SDS).

In one embodiment of any aspect herein, contacting is in vivo, in vitro, or ex vivo.

An edited cell produced by any of the methods described herein.

A composition of edited cells described herein.

In one embodiment of any aspect herein, the composition further comprises a pharmaceutically acceptable carrier.

A method of treating Shwachman-Diamond-Syndrome (SDS), the method comprising administering to the subject in need thereof any of the base editing systems described herein.

A method of treating Shwachman-Diamond-Syndrome (SDS), the method comprising administering to the subject in need thereof an edited cell produced by any of the methods, any of the edited cells, or any of the compositions described herein.

In one embodiment of any aspect herein, the subject comprises a T258C mutation.

In one embodiment of any aspect herein, the edited cell was obtained from the subject in need thereof prior to editing.

In one embodiment of any aspect herein, the edited cell is autologous or allogeneic to the subject in need thereof.

In one embodiment of any aspect herein, administering is a cell transplant.

In one embodiment of any aspect herein, administering results in the reversal of at least one symptom related to Shwachman-Diamond-Syndrome (SDS).

In one embodiment of any aspect herein, the method further comprises the step of diagnosing the subject as having Shwachman-Diamond-Syndrome (SDS) prior to administering.

In one embodiment of any aspect herein, the method further comprises the step of diagnosing the subject as being at risk of having Shwachman-Diamond-Syndrome (SDS) prior to administering.

In one embodiment of any aspect herein, the method further comprises the step of receiving results of an assay that diagnoses the subject as having Shwachman-Diamond-Syndrome (SDS) prior to administering.

In one embodiment of any aspect herein, the method further comprises the step of receiving results of an assay that diagnoses the subject as being at risk of having Shwachman-Diamond-Syndrome (SDS) prior to administering.

### Definitions

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

As used herein, the term “small molecule” refers to a chemical agent including, but not limited to, peptides, peptidomimetics, amino acids, amino acid analogs, polynucleotides, polynucleotide analogs, aptamers, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

A “nucleic acid”, as described herein, can be RNA or DNA, and can be single or double stranded, and can be selected, for example, from a group including: nucleic acid encoding a protein of interest, oligonucleotides, nucleic acid analogues, for example peptide-nucleic acid (PNA), pseudo-complementary PNA (pc-PNA), locked nucleic acid (LNA) etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc.

As used herein, the term “genetic engineered cell” refers to a cell that comprises at least one genetic modification, as that term is used herein.

As used herein, the term “genetic modification” refers to a disruption at the genomic level resulting in an alteration in SBDS gene expression or activity in a cell. Exemplary genetic modifications can include deletions, frame shift mutations, point mutations, exon removal, etc.

By “inhibits SBDS gene expression” is meant that the amount of expression of SBDS gene is at least 5% lower in a cell or cell population treated with a base editor system described herein, than a comparable, control cell or cell population, wherein no base editor system is present. It is preferred that the percentage of SBDS gene expression in a treated population is at least 10% lower, at least 20% lower, at least 30% lower, at least 40% lower, at least 50% lower, at least 60% lower, at least 70% lower, at least 80% lower, at least 90% lower, at least 1-fold lower, at least 2-fold lower, at least 5-fold lower, at least 10 fold lower, at least 100 fold lower, at least 1000-fold lower, or more than a comparable control treated population in which no base editor system is added. At a minimum, SBDS gene activity or expression can be assayed using techniques standard in the art, including, but not limited to, mass spectrometry, immunoprecipitation, or gel filtration assays. For example, detection of SBDS gene activity can be carried out via RT-qPCR, RT-ddPCR or deep sequencing of cellular transcripts to monitor production of SBDS mRNA. Functional SDS protein can be detected and quantified using standard Western Blot procedures. The physiological consequences of SBDS gene correction can furthermore be assayed via ribosome profiling, where the ratio of mature 60S to the immature 80S precursor form normalizes, and the cellular response is increased proliferation as a consequence of lowered p21 expression.

Methods and compositions described herein require that the SBDS gene is edited (i.e., from a mutation version to wild-type. As used herein, “SBDS ribosome maturation factor (SBDS)” refers to a gene that encodes a highly conserved protein that plays an essential role in ribosome biogenesis. The encoded protein interacts with elongation factor-like GTPase 1 to disassociate eukaryotic initiation factor 6 from the late cytoplasmic pre-60S ribosomal subunit allowing assembly of the 80S subunit. Mutations within this gene are associated with the autosomal recessive disorder Shwachman-Bodian-Diamond syndrome. Sequences for SBDS, also known as SDS, SDO1, SWDS and CGI-97, are known for a number of species, e.g., human SBDS (NCBI Gene ID: 51119) polypeptide (e.g., NCBI Ref Seq NP_057122.2) and mRNA (e.g., NCBI Ref Seq NM_016038.4). SBDS can refer to human SBDS, including naturally occurring variants, molecules, and alleles thereof. SBDS refers to the mammalian SBDS of, e.g., mouse, rat, rabbit, dog, cat, cow, horse, pig, and the like. The human nucleic sequence of SEQ ID NO: 6 comprises the nucleic sequence which encodes SBDS.

SEQ ID NO: 6 shows the complete transcribed genomic sequence of SBDS. Accession: chromosome:GRCh38:7:66987519:66995759:1. In SEQ ID NO:2, the protein coding sequences in underlined and the target nucleotide 258+2 shown as wildtype T is

[symbol]

[symbol]

(SEQ ID NO: 6)
AGACACACTGTGACGGCTGCCTGAAGCTAGTGAGTCGCGGCGCCGCGCACTGGTGGTTGGGTCAGTG
CCGCGCGCCGATCGGTCGTTACCGCGAGGCGCTGGTGGCCTTCAGGCTGGACGGCGCGGGTCAGCCCTGGTTCGCCGGCT
TCTGGGTCTTTGAACAGCCGCGATGTCGATCTTCACCCCCACCAACCAGATCCGCCTAACCAATGTGGCCGTGGTACGGA
TGAAGCGTGCCGGGAAGCGCTTCGAAATCGCCTGCTACAAAAACAAGGTCGTCGGCTGGCGGAGCGGCGTGTGAGTAGCC
CCCTCCCTCGGGCCTGGGCCTGGGCCTGAGCCGTCACCTCCGAGGCGGCCTGTCTCTGCCCAAGTCGAGTGAATGGGCCA
GGCTGGGGTGTTGGCCGGGGAGGAAATGGAACATTCCTGCTGTGAGCATGAGACGTCGCTGTCCGAGCTTGGCGCCTAAG
CCAAGGGTTTCTTCTTTATTTGGTTGGTTCGGATTGGGTTGTTGGTTTGGGGTTTTGTTTTGTTGGTGTCATAAAAGCTG
CAGCCAAGAAATCTCGTAATTGTGGTCCTTTTCCTAGAATAATGATGGCTGAGAACCTAGTCTTACGAATACTGTCATAG
TTGTGGGTACATCTGGTGTCATAGATCTCAGTAGCCCTAGCAGTTTAAAGTGCGTAGTGTCTTCACTGCTGCCATCTAGG
GACTGGCATTCCGTGGCAGCAGCTGTCTGCACCCCACCCCACCCTTGTGTTTCTTACGTCCTCCCATGACATTTTTCTCC
AGTGAGCAAATGGTAAGGCAAATACGGTTCTGAGTTTTGAAAATGTTCCCTCAGGCCGATGCGGGCAGTTCACTTGAGGC
CAGGAGTTCGAGGCCAGCCTGGCCAACATGAAACCCCATCTCTACTAAAAATACAAAGTTAGCCGGGTGTGGTGGCGCAT
GCCTGTAATCCCAGTTACTCAGGAGGCTGAGGCGGGAGAATCACTTGAACCCGGGAGGCTGAGGTTACAGTGACCCGAGA
TCGCGCCATTGCACTCCAGCCTGGGCAAAAACAGTGAAATTCCATCTAGGGGCGGGGGTTGGGGGGTAAGAAAAAGAAAA
CTGCCCTCTACACTAAAGGTCATCAGGGGGATTTGTTGTGTCTTGCCGTTCATGTTGTTGCCATCTCGTATTTAAATGTA
AATGCATGTCCAAGTTTCAAGTATATTCACATAGGACTTTCTCTCCTGCCCTCACAAGGGAAAAAGACCTCGATGAAGTT
CTGCAGACCCACTCAGTGTTTGTAAATGTTTCTAAAGGTCAGGTTGCCAAAAAGGAAGATCTCATCAGTGCGTTTGGAAC
AGATGACCAAACTGAAATCTGTAAGCAGGTGGGTAACAGCTGCAGCATAGCTAACCCTAATAACCATTTATAACGTATTT
GTAGATATATTAAACATTAAAGGCTGTTTTTCTGGAGGAAAGACTAACCAAGCAATAATGTGAACTGCACAGTGTCACTT
CTAATAATAAAGAACTTGGTGGTTTTGTTTGTTTTTTGTGGTTTTTTTTTTTTTTTTTTTGCCAAAACCTCCTGAAATCA
GATTTTCTACTAAATTACCTCATTTTTCTATAAATTGCCCCATTTATAGAATGTGGTATACCTGAAAACTGAATTTGTTT
TCAAGTATTTCATCTTTCAGATCCTTTTTTTTTTGGGTGGGGGGACAGAGTCTTGCTCTGTTGCCCAGGCTGGAGTGCAG
TGCACTTCACTGCACTCAGCTCACTGCAACCTCTGCCTCCTGGATTCAAGCGATTCTCTTGCCTCAGCTTCCAGGGTAGC
TGGGATTACAGGTGCCCACCACCCTGCCTGGCTAATTTTTGGATTTTTAGTAGAGATGGGGTTTCACCATGTTGGCCAGG
TTGGTCTCAAACTCCTGACCTTGGGTGATATGCCCGCCTTAGCCACCCAAAGTGCTGGGATTACAGATGTGAGCCACTGA
GCCCAGCCAAAAGATTCTTGCATCTTTTGGGCAAAGCTCAAACCATTACTTACATATTGATAGCTGGAGAGGATGAAATT
TAATTTTCTCTCCATCCAGTTACTCATTTTTTATGGTTAGTTAATAAATAGTGTGTGATAGAGAAAGATAGTGATTTCTT
AAATGTGTTGGCATTTTTTTAGATTTTGACTAAAGGAGAAGTTCAAGTATCAGATAAAGAAAGACACACACAACTGGAGC
AGATGTTTAGGGACATTGCAACTATTGTGGCAGACAAATGTGTGAATCCTGAAACAAAGAGACCATACACCGTGATCCTT
ATTGAGAGAGCCATGAAGGACATCCACTATTCGGTGAAAACCAACAAGAGTACAAAACAGCAGGTGAGTGGTTTCTCATG
TCATCAAAATATAGCCATGGAAATCAGTTTTCTCTGAAGAAATCATTAAAATAATGGGTCTGGGGCCAGGCACAATGGTT
CATGCCTGTAATCCTAGCACTTTGGGAGCCAAGATGGGAGGATTGCTTGAGGCCTGGAAACAGCCTGGGAAACATAGGGA
CGCCCCATCTCTAAATTTTTTTTTTTTTTTTTTGAGACAGAGTCTTACTCTATTGCCCAGGCTGGAGTGCAGTAGTATGA
TCTCGGCTCACTACAATCTCCACCTCCCGCGTTCAAGCAAGTCTCCTGCCTCAGCCTCCTGAGTAGCTGGGATTATAGGC
ACGTGCCACCACACTCAGCTAATTTTGTATTTTTAGTAGAGTTGAGGTTTCACCATGTTGGCCAGGCTGGTCTTGAACTC
CTGACCCTAGGTGATCCGTCCGCCTTGGCCTCCCAAAGTGCTGGGATTACAGGCATCAGCTACCGTACCCTACCTCTAAA
TTTTTTAATATAAAAAATTAAATTTAAAAAAATGGGTCTGCATGGAAGCAAGTGGGTAGAGGTTTGGGGAAGAGATGGAG
GAAAAGGACGGGGTTGTAAAGATAGGTAATTTTTTTTTTTCAAGTAAACACAAATATGTATATAGGACTTTCTTCTTCCT
ATCCAAATCAAACTTTTAAAGACAAAACTTGAGTTTTTAAAGGGTCATTTTAACACTTCTTTTTGAATTTTTTAATTTAT
ATATAATTCACATACCATAAATTTCACACTCATAAAGTATGTACACTTTAAGTGGTATATTAACAAAGTTTTGGAACCTT
CCCTGCTACCTGGTTCGAGAACATTTTCATCACCACAAAAAGAAAGTCAGTATCCATTAGTAGCCATCCCCCATTTTCCC
CCCACAGGCCCCTCCCAACCACTAATCTCCTCTCGTTATGGACTTCTCAATTCTGGACATTTCATATAAATGGAATCATA
CAATATGTGGCCTTTTCATGGTTCATACATGTTGTAACCTGCATCAGCATGTCATTTCTTTTTTATGCCGGAATAATAGC
CCACTGTACGGAAAGAAACACATTTTGTTCATTCATCTATCAGTTGATAGACATTGGGTTGCTTTCACTTTTGAGCTATG
ATGAGCAATGCTGCTATAAAATTTCTTGTATGTTTCTGTGTAGACATATGTTTTCATTTCTGTATACCTGGTGACTACCA
AACCTATTTCTAAAACAGCTGCACCATTTTACTTTACCACCATCAGTGTTTAAGAGTTCAGTTTCTCCACATCCTCAGTA
ATACTTGTCATTGTCTGCCTTTTTGATGATGGCCATCCTGGTGGTATCTTGTCGTGGTTTTGATTTGCATTTCCTTAATG
ATGATTTGAGCATATTTCCATGTGCTTATTGGTGCCTCGTCTGTCTTCTTTTGAGAAATCTCTGTTCAGGTTCTTTGCCC
ACCCCCCCCGCCCTCTTTTTGCAAACTCTGCCTCCCGGATTCAAGCAATTCTCCTGCCTCAGCCTCTTGAGTAGCTGGGA
TTACAGGCGTGCACTACCACACCCGGCTAATTTTTCTTTTTTTGTATTTTTAGTGGAGACGGGGTTTCACCATGTTGGCC
AGGCTGGTCTCGAATTCCTGACCTTGTGATGCACCCGCCTCGGCCTCCCAAAGTGCTGGAATTACAGGCGTGAGCCACCA
CACCTGGCCTTCACTTTCTTCATAGTTTTTTGAAACACAAAAGCTTTTCTTCTTGATAAGTCCAATTTTTCTATTTTTTT
TTTAACGGTCACTTATGTTCTTAATGTTATACCTAAGAAACCATTACCTAATCCAACTACATGGAAACTACTTTGTTTTT
GAAAACCTTATGAAATAATATAGTAGAAGAAATTGCATTCTCGATTTTGTCTTGGTAGGCTTTGGAAGTGATAAAGCAGT
TAAAAGAGAAAATGAAGATAGAACGTGCTCACATGAGGCTTCGGTTCATCCTTCCAGTCAATGAAGGCAAGAAGCTGAAA
GAAAAGCTCAAGCCACTGATCAAGGTCATAGAAAGTGAAGATTATGGCCAACAGTTAGAAATCGTAAGAGTCAAATATTT
TCTTTGCTTCATGTTACCTAAATATTGTATTCTCTAGTAATAAATTTGTAGCAAACATTTAGATGTTGTAAACGTCAGAT
ATTTTCTTTTCTTTTTTTTTTTTGAGATGGAGTCTCGCTCTGTTGCCCAGGCTGGAGTGCAGTGGTGCTATCTCAGCTCA
CTGCAAGCTCCGCCTCCCGGGTACACGCCATTCTCCTGCCTCAGCCTCCTGTGTAGCTGGGACTACAGGCACCCGCCACC
AAGCCTGGCTAATTTTTCGTATTTTTTAGTAGAGACGGAGTTTCACCGTGTTAGCCAGGACGGTCTTGATCTCCTGACCT
CATGATCCACCTGCCTCAGCCTCCCAAAGTGCAGGGATTACAGGCGTGAGCCACCGCGCCCAGCCAGATATTTTCTTAAG
ACTAAAGGTTGGTCAGCCTTGTTTACCACAAATCCTTTTATTTTTTTCCCAGCATTTATTTTTGTCATATATTATGCGTA
GTGTTTTAGATACATGATTTTAGCAAATTTAGCAACTATTTAAAATAAGCCACTTAAAAATTGCTATTTTAGTGGAAGAT
TGATAATTCCAATTACCTTAAAAATTTTGCAAAAATTAGTACAAAGCACACTATGGAAATTTACATTGAGTTTTCAGAGA
TAGCTCAATTGCATGCTTTACTCCAGTGCTGATCATAATCCTAAAAGACAATCCCAAATGCCACAATCCCAATGTTGAAA
TCATAAAAAAATCAAAATCTCTAAAGTCCAAAGCCTGAACTTCTAATATCTTGAAAATCACAGTTCCAAAAGATTGAATC
CTGGCTGGGCGCGGTGGCTCATGCCTGTAATCCCAGCACTTTGGGAGGCCAAGGCAGGTGGATCACCTGAGGTCGGGAGT
TCGAGACCAGTCTGACCAACATGGAGAAACCCTGTCTCTACTAAAAATACAAAATAGTCTGGCGTGGTGGCACATGCCTG
TAATCCCAGCTACTCGGGAGGCTGAGGCAGGAGAATCACTTGAACCTGGAAGGCAGAGGTTGCGGTGAGCCAAGATTGCA
CCATTGCACTCCAGCCTGGGCAACAAGAGCGAAACTCCATCTCAAAAAAAAAAAAAAAAGATTGAATTCTGAATGATGAA
ATTCTGAAAGCTGAATTCTGGCAAAAGGATTAGCACTCTTTGAGTTGTATGCAGGATAGTCGCGTCTGGTTAATTGCATC
ATGTGAGGAAAAGCTAGGACCTTGTTATTGTCTTCATTTGAAAATTAAAAATGGTTTAAAGAGATGAGTCTGGGTGCCAA
GTTGATAAGGGGTGGAATTGTGGATGTAATTTTAGGTGTCAACTTGACTGGAAAGGAATCCCTAGAAGCCTGATAAAGCA
TTGTTTGGGGTATGTCTGTGAGGGTGTTTCCAGAGGATATTAGTGTGTGAACCTGGTGACACTAAACACAGAACATTTTA
AAGAAGCAGCGAAACCATTATGTCCTTATGACCTCACCCCACAGCTTCAAACACAGTGTAAATTGAGCTGTCTACAAATT
CATGTTTTCAAGGCTTTGCAGCTTATTTATTTATTTATTTATTTATTTATTTTTGAGACAGGGTTTCACTCTTATTGCCC
AAGCTAGAGTGCAATGGCGTGATCTCAGCTCACTGCAACCTCTGCCTCCCGGGTTCAAGCGATTCTCCTGCCTCAGCCTC
CCGAGTAGCTGGGATTACAGGCATGTGCCACCTCGCCCAGCTAATTTTGTATTTTTAGTAGAGACGGGGTCTCTCCATGT
TGGTCAGGCTGGTCTCGAACTCCCGACCTCAGGTGATCCACCCGCCTCGGCCTCCCAAAGTGCTGGGATTACAGGCTTGA
GCCACCGCGCCCTGTCTGCAGCTTCTTTTTAAAAGTAAATGAGCTGTAGAGAAGTGTTGAGTATGTGAGTATCCACTGTA
GATGTGAACTAACTCATCTGACACTACTTGAAGTTCTAAAATCTTTGCAAAACTGTACACATGGGCCAGGCACAGTGGCT
CGTGCCTGTAATCCCAGCACTTTGGGAGGCCAAGGTGAGCAGATAACATGGTGAAACCCTATCTCTACTAAAAATACAAA
AAATAAGCCAGGTGTGGTGGTGGGTTCCTGTAATCCCAGTTTCTTGGGAGGCTGAGGCAGGAGAATCACTTGAACCTGGG
AGGCGGAGGCTGCAGTGAGCCAAGATCACACCACTGCACTCTATCTCAAAAAAAAATAAATTAACATACACATGGTGTCT
ACATAAGTCTTCACATTGCTTTTTCTCCTTCATACGTGGAGGTGACTTTACTGAGCTATAAAATGTAATGCTAAATTTTA
GTATGAGAAGAATCAGAGTTTTCTAGTTTGTCCCTTCCATTTACAGCTGAAGAATCAGAATAAGTGTTTAAACATAGGGA
TTAATGCCTTGTCACAGGGGGCTACATGGACACTTGAGGGCAGAGGCTAAACTGGAACCCAGTGTGCCGCCCTACCCATT
GTCTTATCTATTGCACCATAGAACTGTGGTATTATTAGAGATCTGGACAGCATTGTGCTTGCCTCAAAGGAAGTTAAAGC
TGAGTTTATTCTGTGTCTTGCTCATCCTCATGTGGTAATCTGCTACGTTAAATGTTTCAGGTATGTCTGATTGACCCGGG
CTGCTTCCGAGAAATTGATGAGCTAATAAAAAAGGAAACTAAAGGCAAAGGTTCTTTGGAAGTACTCAATCTGAAAGATG
TAGAAGAAGGAGATGAGAAATTTGAATGACACCCATCAATCTCTTCACCTCTAAAACACTAAAGTGTTTCCGTTTCCGAC
GGCACTGTTTCATGTCTGTGGTCTGCCAAATACTTGCTTAAACTATTTGACATTTTCTATCTTTGTGTTAACAGTGGACA
CAGCAAGGCTTTCCTACATAAGTATAATAATGTGGGAATGATTTGGTTTTAATTATAAACTGGGGTCTAAATCCTAAAGC
AAAATTGAAACTCCAAGATGCAAAGTCCAGAGTGGCATTTTGCTACTCTGTCTCATGCCTTGATAGCTTTCCAAAATGAA
AGTTACTTGAGGCAGCTCTTGTGGGTGAAAAGTTATTTGTACAGTAGAGTAAGATTATTAGGGGTATGTCTATACAACAA
AAGGGGGGGTCTTTCCTAAAAAAGAAAACATATGATGCTTCATTTCTACTTAATGGAACTTGTGTTCTGAGGGTCATTAT
GGTATCGTAATGTAAAGCTTGGATGATGTTCCTGATTATCTGAGAAACAGATATAGAAAAATTGTGCCGGACTTACCTTT
CATTGAACATGCTGCCATAACTTAGATTATTCTTGGTTAAAAAATAAAAGTCACTTATTTCTAATTCTTAAAGTTTATAA
TATATATTAATATAGCTAAAATTGTATGTAATCAATAAAACCACTCTTATGTTTATTAAACTATGGCTTGTGTTTCTAGA
CAA

As used herein the term “cleaves” generally refers to the generation of a double-stranded break in the DNA genome at a desired location.

As used herein, the terms “adenine”, “guanine”, “cytosine”, “thymine”, “uracir” and “hypoxanthine” (the nucleobase in inosine) refer to nucleobases.

As used herein, the terms “adenosine”, “guanosine”, “cytidine”, “thymidine”, “uridine” and “inosine”, refer to the nucleobases linked to the (deoxy)ribosyl sugar.

As used herein, the term “effective amount of a base editing system” refers to an amount of base editing system described herein that yields sufficient base editing activity to generate a base substitution without a double-stranded break in the desired location of the genome, e.g., nucleotide 258 of the SBDS gene. In one embodiment, the effective amount of the base editing system induces a base substitution at the desired genetic locus in at least 20% of the cells in a population contacted with the composition (e.g., at least 30%, at least 40%, at least 50%, at least 60%, at least 70%, at least 80%, at least 90%, at least 95%, at least 99%, or even 100% of the cells in the population comprise a genetic modification produced by the base editing system or composition thereof).

The term “isolated cell” as used herein refers to a cell that has been removed from an organism in which it was originally found, or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched. In some embodiments, the isolated population is an isolated population of cells, e.g., a substantially pure population of human bone marrow cells as compared to a heterogeneous population of cells comprising human bone marrow cells and cells from which the human bone marrow cells were derived.

The term “substantially pure,” with respect to a particular cell population, refers to a population of cells that is at least about 75%, preferably at least about 85%, more preferably at least about 90%, and most preferably at least about 95% pure, with respect to the cells making up a total cell population. That is, the terms “substantially pure” or “essentially purified,” with regard to a population of, e.g., bone marrow cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not bone marrow cells as defined by the terms herein.

A “subject,” as used herein, includes any animal that exhibits a symptom of a monogenic disease, disorder, or condition that can be treated with the cell-based therapeutics, and methods disclosed elsewhere herein. In preferred embodiments, a subject includes any animal that exhibits symptoms of a disease, disorder, or condition of the pancrease, bone marrow or bone, e.g., Shwachman-Diamond syndrome (SDS), that can be treated with the gene therapy/cell-based therapeutics, and methods contemplated herein. Suitable subjects (e.g., patients) include laboratory animals (such as mouse, rat, rabbit, or guinea pig), farm animals, and domestic animals or pets (such as a cat or dog). Non-human primates and, preferably, human patients, are included. Typical subjects include animals that exhibit aberrant amounts (lower or higher amounts than a “normal” or “healthy” subject) of one or more physiological activities that can be modulated by gene therapy.

In one embodiment, as used herein, “prevent,” and similar words such as “prevented,” “preventing” etc., indicate an approach for preventing, inhibiting, or reducing the likelihood of the occurrence or recurrence of, a disease or condition. In another embodiment, the term refers to delaying the onset or recurrence of a disease or condition or delaying the occurrence or recurrence of the symptoms of a disease or condition. In another embodiment, as used herein, “prevention” and similar words includes reducing the intensity, effect, symptoms and/or burden of a disease or condition prior to onset or recurrence of the disease or condition.

As used herein, the term “treating” includes reducing or alleviating at least one adverse effect or symptom of a condition, disease or disorder. For example, the term “treating” and “treatment” refers to administering to a subject an effective amount of a composition, e.g., an effective amount of a composition comprising a population of cells, e.g., bone marrow cells, so that the subject has a reduction in at least one symptom of the disease or an improvement in the disease, for example, beneficial or desired clinical results. For purposes of this disclosure, beneficial or desired clinical results include, but are not limited to, alleviation of one or more symptoms, diminishment of extent of disease, disease stabilization (e.g., not worsening), delay or slowing of disease progression, amelioration or palliation of the disease state, and remission (whether partial or total), whether detectable or undetectable. In some embodiments, treating can refer to prolonging survival as compared to expected survival if not receiving treatment. Thus, one of skill in the art realizes that a treatment can improve the disease condition, but may not be a complete cure for the disease. In some embodiments, treatment can include prophylaxis. However, in alternative embodiments, treatment does not include prophylaxis.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used with the methods described herein that will be effective in the treatment of a particular disorder or condition will depend on the nature of the disorder or condition, and can be determined by standard clinical techniques.

As used herein, “prevention” or “preventing,” when used in reference to a disease, disorder or symptoms thereof, refers to a reduction in the likelihood that an individual will develop a disease or disorder, e.g., SDS. The likelihood of developing a disease or disorder is reduced, for example, when an individual having one or more risk factors for a disease or disorder either fails to develop the disorder or develops such disease or disorder at a later time or with less severity, statistically speaking, relative to a population having the same risk factors and not receiving treatment as described herein. The failure to develop symptoms of a disease, or the development of reduced (e.g., by at least 10% on a clinically accepted scale for that disease or disorder) or delayed (e.g., by days, weeks, months or years) symptoms is considered effective prevention.

The term “mammal” is intended to encompass a singular “mammal” and plural “mammals,” and includes, but is not limited to humans; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and bears. In some preferred embodiments, a mammal is a human.

In one embodiment, the term “effective amount”, as used herein, refers to the amount of a cell composition that is safe and sufficient to treat, lesson the likelihood of, or delay the development of a SDS. The amount can thus cure or result in amelioration of the symptoms of SDS, slow the course of SDS progression, slow or inhibit a symptom of SDS, slow or inhibit the establishment of secondary symptoms of SDS or inhibit the development of a secondary symptom of SDS. The effective amount for the treatment of SDS depends on the severity of the symptoms, the subject being treated, the age and general condition of the subject, the mode of administration and so forth. Thus, it is not possible or prudent to specify an exact “effective amount”. However, for any given case, an appropriate “effective amount” can be determined by one of ordinary skill in the art using only routine experimentation.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### BRIEF DESCRIPTION OF DRAWINGS

This application file contains at least one drawing executed in color. Copies of this patent application publication with color drawings will be provided by the Office upon request and payment of the necessary fee.

FIGS. 1A and 1B show base editing to correct the most common SBDS 258+2T>C mutation in Shwachman-Diamond Syndrome. FIG. 1A) HEK293T SBDS-mCherry reporter cell line which includes wildtype intron or the 258+2T>C mutation designed to facilitate the screening of successful base editing. FIG. 1B) mCherry fluorescence intensity is greatly reduced in clonally derived reporter lines when the intronic SDS mutation is present.

FIGS. 2A and 2B show screening of the most efficient base editor combination to correct SBDS 258+2T>C mutation in reporter cells. FIG. 2A) Multiple base editors and sgRNAs were screened in our SBDS-mCherry reporter cell line by detecting mCherry fold change via flow cytometry. FIG. 2B) Sanger sequencing of the most efficient combination in A (Orange bar) shows high C to T editing rate in both 258+2C and −4 bystander C, which introduces a loss of function stop codon. This needs to be avoided.

FIGS. 3A-3E show shifting the sgRNAs to avoid bystander editing of −4C. FIG. 3A) sgRNAs were shifted to exclude −4 bystander C to increase functional correction. FIGS. 3B-3E) Multiple different base editors and shifted sgRNAs were screened in mCherry reporter cell line by detecting mCherry fold change via FACS. The bold framed box in Fig. D indicates the best combination (evoFERNY-SpRY with the target C in position 3 of the sgRNA and 20 nt total sgRNA length).

FIGS. 4A-4C show an alternative strategy of imperfect sgRNAs (isgRNA) to avoid edits on bystander −4C. FIG. 4A) sgRNAs with mismatches on −4 bystander C were tested with the aim of increasing functional correction (high on-target with minimal bystander editing). FIG. 4B) Multiple base editors and shifted sgRNAs were screened in mCherry reporter cell line.

Functional editing is calculated by on target editing rate minus bystander editing rate. FIG. 4C) Functional editing measured by sanger sequencing of shifted sgRNA vs isgRNA in mCherry reporter cell line indicates that isgRNAs perform superior.

FIGS. 5A-5C show base editing on 258+2T>C mutation with optimized base editor and shifted sgRNA recovers WT splicing of SBDS mRNA. FIG. 5A) The most efficient combination from the screen (evoFERNY-SpRY with C in position 3 and a 20 nt sgRNA) shows 25% C to T editing on 258+2C without editing on −4 bystander C in 293T cells. FIG. 5B) evoFERNY-SpRY+C3-20 bp resulted in high editing rate in SDS patient derived fibroblasts. FIG. 5C) Correction of SBDS 258+2T>C mutation via base editing resulted in reconstitution of wildtype splicing of SBDS mRNA in SDS patient derived fibroblasts to −40% of wildtype. Since editing rate on the genomic level was 33% this suggests complete reconstitution of mRNA expression in each edited cell.

FIG. 6 shows no detectable off-target editing on predicted target sites. The four highest-scoring relevant off-target sites predicted in silico with CRISPRme software were assessed via amplicon sequencing in SDS patient-derived fibroblasts. No significant off-target editing was observed.

FIGS. 7A and 7B show pptimization of VLP mediated base editor delivery into patient cells. FIG. 7A) Delivery of evoFERNY-SpRY+isgRNA via virus-like particles (VLP) was optimized by testing repeat transduction over three consecutive days. FIG. 7B) The same procedure on healthy donor CD34 cells indicates higher editing with each successive round of editing via VLP.

FIGS. 8A-8D show correcting the SDS mutation in primary patient derived bone marrow cells. FIG. 8A) Correction of primary SDS patient-derived mononuclear bone marrow cells with homozygous 258+2 T>C mutation using evoFERNY-SpRY+isgRNA delivered via virus like particles (VLP). FIG. 8B) Hiability after gene correction highlighting the compatibility of base editing via VLP to modify primary bone marrow cells. FIG. 8C) Increased proliferation of corrected cells as a consequences of improved cells fitness. FIG. 8D) Gene correction is associated with reduced CDKN1A expression, a major regulator cell proliferation and a hallmark of bone marrow failure syndromes.

### DETAILED DESCRIPTION

### Shwachman-Diamond-Syndrome (SDS)

Shwachman-Diamond syndrome (SDS) is a rare inherited disorder resulting from a mutation in the SBDS gene occurring in approximately 1 out of every 75,000 births. Approximately 90% of all individuals diagnosed with SDS carry a mutated version of the SBDS gene. Such mutation can be inherited from both biological parents (autosomal recessive) or from one parent combined with a newly arising mutation. SDS is most commonly diagnosed before a subject's first birthday and primarily affects children's pancreases, bone marrow and bones. SDS is complicated and does not always result in the same symptoms; e.g., one child may have pancreas and skeletal issues while another may have bone marrow issues and skeletal. SDS symptoms may be mild or severe, and may change over time. Subjects disagonosed with SDS commonly require life-long medical care, but typically have normal lifespans. Subject having SDS may also have an increased risk of developing of life-threatening blood cancers or serious blood disorders. Individuals with SDS have an increased risk of developing abnormal blood cells (myelodysplasia) that may become acute myeloid leukemia, as compared to an unaffected individual.

SDS may cause several medical problems, with the three most common being exocrine pancreatic insufficiency, impaired bone marrow function, and skeletal abnormalities. SDS can result in exocrine pancreatic insufficiency. For example, the pancreatic acinar cells fail to produce enough enzymes to break down food, preventing nutrient absorption, as compared to an unaffected individual.

SDS can result in impaired bone marrow function. For example, the bone marrow produces a decreased amount of red blood cells, white blood cells and/or platelets as compared to bone marrow from an unaffected individual. The bone marrow further produces a decreased amount of neutrophils as compared to bone marrow from an unaffected individual. Accordingly, a subject having SDS may be more susceptible to a microbial infection, for example, pneumonia, middle ear infections (otitis media) or skin infections (cellulitis), as compared to an unaffected individual.

SDS affects several parts of the body, most commonly the pancreas, bone marrow and skeletal system. Symptoms may be present at birth, during infancy, as young children and as young adults. The most common symptoms include failure to thrive (failure gain weight as a baby); fatigue; irritable; lethargic; Large, greasy bowel movements; serious and reoccurring microbial infections; Noticeable shortened arm and/or leg bones.

SDS is diagnosed by a healthcare providers via assays well established in the art. For example, diagnosis can be determined by a physical examinations to evaluate overall health; complete blood count (CBC) with differential (i.e., to measures and counts blood cells, including all white blood cells); pancreas function tests (i.e., via a stool sample or imaging tests such as computed tomography (CT) scans); Blood tests to check vitamin levels; X-rays to look for signs of skeletal problems (particularly in your child's hips or lower limbs); and/or genetic testing to determine if common mutations in the SBDS gene are present.

Current therapeutics for SDS include, but are not limited to blood transfusions, e.g., to increase blood cell levels; platelet transfusions, e.g., to increase platelet levels; granulocyte-colony stimulation factor (G-CSF); e.g., to increase the number of neutrophils; stem cell transplantation; and/or orthopedic surgery, e.g., to correct severe skeletal problems.

As used herein, treating or reducing a risk of developing a SDS in a subject means to ameliorate at least one symptom of SDS. In one aspect, the invention features methods of treating, e.g., reducing severity or progression of, a SDS in a subject. In another aspect, the methods can also be used to reduce a risk of developing a SDS in a subject, delaying the onset of symptoms of a SDS in a subject, or increasing the longevity of a subject having a SDS. In one aspect, the methods can include selecting a subject on the basis that they have, or are at risk of developing, a SDS, but do not yet have a SDS, or a subject with an underlying SDS. Selection of a subject can include detecting symptoms of a SDS, a blood test, genetic testing, or clinical recordings. If the results of the test(s) indicate that the subject has a SDS, the methods also include administering the compositions described herein, thereby treating, or reducing the risk of developing, a SDS in the subject.

By the phrase “risk of developing disease” is meant the relative probability that a subject will develop a SDS in the future as compared to a control subject or population (e.g., a healthy subject or population). For example, an individual carrying the genetic mutation associated with SDS, an T to C mutation of the SBDS gene, and whether the individual in heterozygous or homozygous for that mutation increases that individual's risk.

As used herein, the term “genome editing” refers to a reverse genetics method using artificially engineered nucleases to cut and create specific double-stranded breaks at a desired location(s) in the genome, which are then repaired by cellular endogenous processes such as, homologous recombination (HR), homology directed repair (HDR) and non-homologous end-joining (NHEJ). NHEJ directly joins the DNA ends in a double-stranded break, while HDR utilizes a homologous sequence as a template for regenerating the missing DNA sequence at the break point.

### Base Editing System

Provided herein is a base editor system for editing the SBDS gene comprising a base editor protein and a guide polynucleotide, wherein the base editor protein comprises a polynucleotide programmable DNA binding domain and a cytosine deaminase domain.

Also provided herein is a base editor system comprising a base editor protein and a guide polynucleotide, wherein the base editor protein is evoFERNY-SpRY and the guide polynucleotide has sequence of SEQ ID NO: 1

As described herein, a “base editor” refers to a genome editing system that induces a base substitution, e.g., an A to a T, or a G to a C, but does not induce double stranded breaks. Cas nucleases, or other endonucleases with CRISPR genome editing are not base editors.

RNA editing is a co- or post-transcriptional process that alters transcript sequences without any change in the encoding DNA sequence. Although various types of RNA editing have been observed in single cell organisms to mammals, base modifications by deamination of adenine to inosine (A>I), or cytidine to uracil (C>U) are the major types of RNA editing in higher eukaryotes. I and U are read as guanosine (G) and thymine (T) respectively by the cellular machinery during mRNA translation and reverse transcription. In contrast to gene-editing nucleases, base editors do not introduce double-strand breaks or exogenous donor DNA templates, and induce lower levels of unwanted variable-length insertion/deletion mutations (indels).

In one embodiment, the polynucleotide programmable DNA binding domain is a SpRY variant of a Streptococcus pyogenes Cas9 (SpCas9).

In one embodiment, the base editing system comprises at least one of the cytosine deaminase domains listed in Table 1A, and at least one of the guide polynucleotides listed in Table 1B.

TABLE 1A
cytosine deaminase domain

evoCDA1-SpG
evoCDA1-SpRY
evoAPOBEC1-SpG
evoAPOBEC1-SpRY
evoFERNY-SpG
evoRERNY-SpRY

TABLE 1B
guide polynucleotide

5′-ATAGGCGGGTAACAGCTGCA (SEQ ID NO: 1)
5′-GGCGGGTAACAGCTGCAGCAT (SEQ ID NO: 2)
5′-AGGCGGGTAACAGCTGC (SEQ ID NO: 3)
5′-GGCGGGTAACAGCTGCAGCA (SEQ ID NO: 4)
5′-AGGCGGGTAACAGCTGC (SEQ ID NO: 5)

In one embodiment, the cytosine deaminase domain is selected from the group consisting of evoCDA1-SpG; evoCDA1-SpRY; evoAPOBEC1-SpG; evoAPOBEC1-SpRY; evoFERNY-SpG; and evoRERNY-SpRY. In one embodiment, the cytosine deaminase domain is evoFERNY-SpRY.

In one embodiment, the guide polynucleotide is selected from:

(SEQ ID NO: 1)
5′-ATAGGCGGGTAACAGCTGCA
(SEQ ID NO: 2)
5′-GGCGGGTAACAGCTGCAGCAT
(SEQ ID NO: 3)
5′-AGGCGGGTAACAGCTGC
(SEQ ID NO: 4)
5′-GGCGGGTAACAGCTGCAGCA
(SEQ ID NO: 5)
5′-AGGCGGGTAACAGCTGC

In one embodiment, the cytosine deaminase domain is an evoFERNY-SpRY and the guide polynucleotide comprises a nucleic acid sequence of SEQ ID NO: 1.

In one embodiment, the cytosine deaminase domain is an evoCDA1-SpG and the guide polynucleotide comprises a nucleic acid sequence selected from SEQ ID NO: 2-4.

In one embodiment, the cytosine deaminase domain is an evoCDA1-SpRY and the guide polynucleotide comprises a nucleic acid sequence selected from SEQ ID NO: 2-4.

In one embodiment, the cytosine deaminase domain is an evoAPOBEC1-SpG and the guide polynucleotide comprises a nucleic acid sequence of SEQ ID NO: 5.

In one embodiment, the cytosine deaminase domain is an evoAPOBEC1-SpRY and the guide polynucleotide comprises a nucleic acid sequence of SEQ ID NO: 5.

In one embodiment, the cytosine deaminase domain is an evoFERNY-SpG and the guide polynucleotide comprises a nucleic acid sequence of SEQ ID NO: 5.

In one embodiment, the cytosine deaminase domain is an evoRERNY-SpRY and the guide polynucleotide comprises a nucleic acid sequence of SEQ ID NO: 5.

In one embodiment, the base editor is an A>I base editor. In one embodiment, the base editor is an C>U or C>T base editor. RNA-dependent ADAR1, ADAR2 and ADAR3 adenosine deaminases, and APOBEC1 cytidine deaminase are non-limiting examples of RNA base editors known in mammals. RNA sequencing studies suggest that A>I RNA base editing affects hundreds of thousands of sites, though most of A>I RNA edits occur at a low level and in non-coding intronic and untranslated regions, especially in the context of specific sequences such as Alu elements. A>I editing of protein-coding RNA sequences at a high level (>20%) is rare and thought to occur predominantly in the brain.

In one embodiment, the base editor is any known base editor, e.g., a third generation base editor, A3A (N57Q)-BE3, A3A-BE3, or A3A (N57G)-BE3, or any yet to be discovered based editor. Base editors are further described in, e.g., international application PCT/EP2017/065467; and U.S. patent application U.S. Ser. No. 10/934,090 and U.S. Ser. No. 15/564,984, which are incorporated herein by reference in their entireties.

Unlike A>I editing catalyzed by adenosine deaminases, the prevalence and level of C>U RNA editing in different types of cells, and its enzymatic basis and regulation are poorly understood. Exemplary C>U base editors include, but are not limited to, activation-induced deaminase (AID), apolipoprotein B editing catalytic polypeptide-like (APOBEC) family, and cytidine deaminase (CDA). In one embodiment, the C>U base editor is any proteins harboring the cytidine deaminase motif for hydrolytic deamination of C to U. CDA is involved in the pyrimidine salvaging pathway. While AID causes C>U deamination of DNA, multiple studies have failed to identify any RNA editing activity for this protein. The base editor of this invention can be selected from any of the 10 APOBEC genes (APOBEC1, 2, 3A-D, 3F-H and 4) identified in humans. APOBEC3 proteins can deaminate cytidines in single-stranded (ss) DNA, and although the APOBEC proteins bind RNA, C>U deamination of RNA is known for only APOBEC1, with apolipoprotein B (APOB) mRNA as its physiological target. C>U RNA editing alters hundreds of cytidines in chloroplasts and mitochondria of flowering plants.

Mutations in the cytidine deaminase enzyme can shorten the length of the editing window and thereby partially address off target editing. In one embodiment, the base editor is mutated to reduce the length of editing window (e.g., the region along the gene sequence which the base editor can target). In one embodiment, the editing window is reduced by at least 1, 2, 3, 4, 5, 6, 7, 8, 9, 10, 11, 12, 13, 14, 15, 16, 17, 18, 19, 20, 21, 22, 23, 24, 25, or more base pairs as compared to the editing window of a wild-type base editor.

In one embodiment, the natural diversity of cytidine deaminases is leveraged to identify one with greater sequence specificity than the rat APOBEC1 (rAPO1) deaminase present in the widely used BE3 architecture. BE3 consists of a Streptococcus pyogenes Cas9 nuclease bearing a mutation that converts it into a nickase (nCas9) fused to rAPO1 and a uracil glycosylase inhibitor (UGI). In one embodiment, rAPO1 is replaced in BE3 with the human APOBEC3A (A3A) cytidine deaminase to create A3A-BE3.

As shown in Gehrke, J M, et al. Nature Biotech. 30 Jul. 2018, the contents of which is incorporated herein by reference in its entirety, base editors can be mutated to induce greater precision within the editing window. In one embodiment, the base editor is mutated to increases its precision and efficiency of gene editing within the editing window. In one embodiment, the base editor's precision and/or efficiency is increased at least 5%, 10%, 15%, 20%, 25%, 30%, 35%, 40%, 45%, 50%, 55%, 60%, 65%, 70%, 75%, 80%, 85%, 90%, 95%, 99%, or more as compared to a wild-type base editor. One skilled in the art can assess the precise and/or efficiency of a base editor by performing, e.g., genome sequencing of a cell targeted by a base editor and compare it to the sequence of the cell prior to editing by the base editor.

In one embodiment, the SBDS gene comprises a mutation. In one embodiment, the SBDS gene comprises a T258C mutation.

In one embodiment, the base editing system edits the SBDS gene at the nucleotide 258.

In one embodiment, the base editing system corrects the T258C mutation in the SBDS gene.

In one embodiment, correcting the T258C mutation in the SBDS gene results in correct splicing of the SBDS gene. In one embodiment, splicing of the SBDS gene is corrected by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more as compared to a cell not contacted by the base editor system.

In one embodiment, correcting the T258C mutation in the SBDS gene results in increased SBDS gene product expression in the cell. In one embodiment, the SBDS gene product expression in the cell is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more, or at least at least 1×, at least 2×, at least 3×, at least 4×, at least 5×, at least 6×, at least 7×, at least 8×, at least 9×, at least 10×, at least 11×, at least 12×, at least 13×, at least 14×, at least 15×, at least 16×, at least 17×, at least 18×, at least 19×, at least 20×, at least 21×, at least 22×, at least 23×, at least 24×, at least 25×, or more as compared to a cell not contacted by the base editor system.

In one embodiment, correcting the T258C mutation in the SBDS gene results in increased cell proliferation. In one embodiment, the cell proliferation is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more, or at least at least 1×, at least 2×, at least 3×, at least 4×, at least 5×, at least 6×, at least 7×, at least 8×, at least 9×, at least 10×, at least 11×, at least 12×, at least 13×, at least 14×, at least 15×, at least 16×, at least 17×, at least 18×, at least 19×, at least 20×, at least 21×, at least 22×, at least 23×, at least 24×, at least 25×, or more as compared to a cell not contacted by the base editor system.

In one embodiment, correcting the T258C mutation in the SBDS gene results in increased ribosome maturation in the cell. In one embodiment, the ribosome maturation in the cell is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more, or at least at least 1×, at least 2×, at least 3×, at least 4×, at least 5×, at least 6×, at least 7×, at least 8×, at least 9×, at least 10×, at least 11×, at least 12×, at least 13×, at least 14×, at least 15×, at least 16×, at least 17×, at least 18×, at least 19×, at least 20×, at least 21×, at least 22×, at least 23×, at least 24×, at least 25×, or more as compared to a cell not contacted by the base editor system.

In one embodiment, correcting the T258C mutation in the SBDS gene results in increased protein synthesis in the cell. In one embodiment, the protein synthesis in the cell is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more, or at least at least 1×, at least 2×, at least 3×, at least 4×, at least 5×, at least 6×, at least 7×, at least 8×, at least 9×, at least 10×, at least 11×, at least 12×, at least 13×, at least 14×, at least 15×, at least 16×, at least 17×, at least 18×, at least 19×, at least 20×, at least 21×, at least 22×, at least 23×, at least 24×, at least 25×, or more as compared to a cell not contacted by the base editor system.

Also provided herein is a method of editing a cell comprising contacting a cell with any of the base editor systems described herein.

Also provided herein is a method of producing a cell having at least one modification in the SBDS gene, comprising contacting a cell with any of the base editor systems described herein.

In one embodiment, the contacting is in vitro, in vivo, or ex vivo.

In one embodiment, contacting occurs for a time sufficient to express the base editor system in the cell. For example, contacting is for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, or at least 1 day, at least 2 days, at least 3 days, at least 4 days, at least 5 days or more. Methods for determining if the base editor system is expressed in the cell are known in the art (for example, western blotting or PCR-based assays) and can be used by a skilled practitioner to determine the time sufficient.

“Contacting” can refer to any known means for expressing the base editing system in the cell. For example, contacting can refer to use of lipid-based carriers (e.g., lipofectamine), electroporation, incubation, infection (e.g., via a viral carrier), etc.

In one embodiment, the method of editing a cell corrects a mutation in the SBDS gene. For example, a mutation in the SBDS gene that causes SDS. In one embodiment, the method of editing a cell corrects, a T258C mutation in the SBDS gene.

In one embodiment of this aspect, the contacted cell acquires at least one genetic modification. For example, the at least one genetic modification is a deletion, insertion or substitution of the nucleic acid sequence. In one embodiment, the at least one genetic modification is a substitution.

In one embodiment, the cell is isolated before contacting.

In one embodiment, the cell is obtained from a subject diagnosed as having Shwachman-Diamond-Syndrome (SDS) or at risk of having Shwachman-Diamond-Syndrome (SDS).

In one embodiment, the cell comprises a mutation in the SBDS gene. For example, a mutation in the SBDS gene that causes SDS. In one embodiment, the cell comprises a T258C mutation in the SBDS gene.

Also provided herein is an edited cell produced using any of the base editor systems described herein, or methods for using the same.

Also provided herein are compositions any of the edited cells described herein. In one embodiment, the composition comprises a pharmaceutically acceptable carrier.

### Compositions and Methods for Treatment

Provided herein is a composition comprising a base editor system for editing the SBDS gene comprising a base editor protein and a guide polynucleotide, wherein the base editor protein comprises a polynucleotide programmable DNA binding domain and a cytosine deaminase domain, wherein the cytosine deaminase domain is an evoFERNY-SpRY and the guide polynucleotide comprises a nucleic acid sequence of SEQ ID NO: 1.

Provided herein is a composition comprising a base editor system for editing the SBDS gene comprising a base editor protein and a guide polynucleotide, wherein the base editor protein comprises a polynucleotide programmable DNA binding domain and a cytosine deaminase domain, wherein the cytosine deaminase domain is an evoCDA1-SpG and the guide polynucleotide comprises a nucleic acid sequence selected from SEQ ID NO: 2-4.

Provided herein is a composition comprising a base editor system for editing the SBDS gene comprising a base editor protein and a guide polynucleotide, wherein the base editor protein comprises a polynucleotide programmable DNA binding domain and a cytosine deaminase domain, wherein the cytosine deaminase domain is an evoCDA1-SpRY and the guide polynucleotide comprises a nucleic acid sequence selected from SEQ ID NO: 2-4.

Provided herein is a composition comprising a base editor system for editing the SBDS gene comprising a base editor protein and a guide polynucleotide, wherein the base editor protein comprises a polynucleotide programmable DNA binding domain and a cytosine deaminase domain, wherein the cytosine deaminase domain is an evoAPOBEC1-SpG and the guide polynucleotide comprises a nucleic acid sequence of SEQ ID NO: 5.

Provided herein is a composition comprising a base editor system for editing the SBDS gene comprising a base editor protein and a guide polynucleotide, wherein the base editor protein comprises a polynucleotide programmable DNA binding domain and a cytosine deaminase domain, wherein the cytosine deaminase domain is an evoAPOBEC1-SpRY and the guide polynucleotide comprises a nucleic acid sequence of SEQ ID NO: 5.

Provided herein is a composition comprising a base editor system for editing the SBDS gene comprising a base editor protein and a guide polynucleotide, wherein the base editor protein comprises a polynucleotide programmable DNA binding domain and a cytosine deaminase domain, wherein the cytosine deaminase domain is an evoFERNY-SpG and the guide polynucleotide comprises a nucleic acid sequence of SEQ ID NO: 5.

Provided herein is a composition comprising a base editor system for editing the SBDS gene comprising a base editor protein and a guide polynucleotide, wherein the base editor protein comprises a polynucleotide programmable DNA binding domain and a cytosine deaminase domain, wherein the cytosine deaminase domain is an evoRERNY-SpRY and the guide polynucleotide comprises a nucleic acid sequence of SEQ ID NO: 5.

One aspect provided herein is a method of treating Shwachman-Diamond-Syndrome (SDS) comprising administering to the subject in need thereof any of the base editing systems described herein.

Another aspect provided herein is a method of treating Shwachman-Diamond-Syndrome (SDS) comprising administering to the subject in need thereof any of the edited cells produced by any method described herein, or any of the edited cells described herein, or any composition thereof.

In one embodiment, the subject comprises a T258C mutation.

In one embodiment, the edited cell to be administered was obtained from the subject in need thereof prior to editing.

In one embodiment, the edited cell to be administered is autologous or allogeneic to the subject in need thereof.

In one embodiment, wherein editing cells or a composition thereof is administered, administering is a cell transplant.

In one embodiment, administering results in the reversal of at least one symptom related to Shwachman-Diamond-Syndrome (SDS). For example, administering results in the reversal of at least T258C mutation in the SBDS gene.

In one embodiment, the method further comprises the step of diagnosing the subject as having Shwachman-Diamond-Syndrome (SDS) prior to administering.

In one embodiment, the method further comprises the step of diagnosing the subject as being at risk of having Shwachman-Diamond-Syndrome (SDS) prior to administering.

In one embodiment, the method further comprises the step of receiving results of an assay that diagnoses the subject as having Shwachman-Diamond-Syndrome (SDS) prior to administering.

In one embodiment, the method further comprises comprising the step of receiving results of an assay that diagnoses the subject as being at risk of having Shwachman-Diamond-Syndrome (SDS) prior to administering.

In one embodiment, the edited cells having at least one genetic modification are not cryopreserved prior to transplanting into mammal for treating SDS.

In one embodiment, the edited cells having at least one genetic modification are cryopreserved, prior to transplanting into mammal for treating SDS.

In one embodiment, the edited cells having at least one genetic modification are cryopreserved, thawed and transplanted into mammal for treating SDS.

In one embodiment, the cells of any compositions described are autologous, to the mammal who is the recipient of the cells in a transplantation procedure, i.e., the cells of the composition are derived or harvested from the mammal prior to any described genetic modification or targeted gene editing.

In one embodiment, the cells of any compositions described are non-autologous to the mammal who is the recipient of the cells in a transplantation procedure, i.e., the cells of the composition are not derived or harvested from the mammal prior to any described genetic modification or targeted gene editing.

In one embodiment, the cells of any compositions described are at the minimum HLA type matched with to the mammal who is the recipient of the cells in a transplantation procedure.

In one embodiment, the cells of any compositions described are isolated cells prior to any described genetic modification or targeted gene editing.

In one embodiment, the method further comprises selecting a mammal in need of treating SDS.

In one embodiment, the mammal has been diagnosed with SDS.

In one embodiment, the contacted cell, human cell, hematopoietic progenitor cell or its progeny is administered to the mammal.

In one embodiment, the method comprises chemotherapy and/or radiation therapy to remove or reduced the endogenous hematopoietic progenitor or stem cells in the mammal.

In one embodiment, the contacted cells, targeted gene edited cells described herein having at least one genetic modification can be cryopreserved and stored until the cells are needed for administration into a mammal.

In one embodiment, the contacted cells, targeted gene edited cells described herein having at least one genetic modification can be cultured ex vivo to expand or increase the number of cells prior to storage, e.g., by cryopreservation, or prior to use, e.g., transplanted into a recipient mammal, e.g., a subject.

In a particular embodiment, a method of preventing, ameliorating, or treating a SDS in a subject is provided. The method comprises administering a population of cells comprising engineered/genetically modified (i.e., edited) cells described herein.

In some embodiments, the population of edited ells can be culture expanded in vitro or ex vivo prior to implantation/engraftment into a subject or prior to cryopreservation for storage.

In some embodiments, the population of edited cells can be culture expanded in vitro or ex vivo after cryopreservation prior to implantation/engraftment into a subject.

In some embodiments of any methods described, the population of edited cells can be differentiated in vitro or ex vivo prior to implantation into a subject.

The genetically modified (i.e., edited) cells may be administered as part of a bone marrow or cord blood transplant in an individual that has or has not undergone bone marrow ablative therapy. In one embodiment, edited cells contemplated herein are administered in a bone marrow transplant to an individual that has undergone chemoablative or radioablative bone marrow therapy.

In one embodiment of any method described, a dose of genetically modified (i.e., edited) cells is delivered to a subject intravenously. In one embodiment, genetically modified hematopoietic cells are intravenously administered to a subject.

In particular embodiments, patients receive a dose of genetically modified (i.e., edited) cells, e.g., hematopoietic stem cells, of about 1×10⁵ cells/kg, about 5×10⁵ cells/kg, about 1×10⁶ cells/kg, about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 5×10⁷ cells/kg, about 1×10⁸ cells/kg, or more in one single intravenous dose. In certain embodiments, patients receive a dose of genetically modified cells, e.g., hematopoietic stem cells described herein or genetic engineered cells described herein or progeny thereof, of at least 1×10⁵ cells/kg, at least 5×10⁵ cells/kg, at least 1×10⁶ cells/kg, at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, at least 1×10⁷ cells/kg, at least 5×10⁷ cells/kg, at least 1×10⁸ cells/kg, or more in one single intravenous dose.

In an additional embodiment, patients receive a dose of genetically modified cells, e.g., hematopoietic stem cells, of about 1×10⁵ cells/kg to about 1×10⁸ cells/kg, about 1×10⁶ cells/kg to about 1×10⁸ cells/kg, about 1×10⁶ cells/kg to about 9×10⁶ cells/kg, about 2×10⁶ cells/kg to about 8×10⁶ cells/kg, about 2×10⁶ cells/kg to about 8×10⁶ cells/kg, about 2×10⁶ cells/kg to about 5×10⁶ cells/kg, about 3×10⁶ cells/kg to about 5×10⁶ cells/kg, about 3×10⁶ cells/kg to about 4×10⁸ cells/kg, or any intervening dose of cells/kg.

In various embodiments, the methods described here provide more robust and safe gene therapy than existing methods and comprise administering a population or dose of cells comprising about 5% transduced/genetically modified (i.e., edited) cells, about 10% transduced/genetically modified cells, about 15% transduced/genetically modified cells, about 20% transduce/genetically modified d cells, about 25% transduced/genetically modified cells, about 30% transduced/genetically modified cells, about 35% transduced/genetically modified cells, about 40% transduced/genetically modified cells, about 45% transduced/genetically modified cells, or about 50% transduce/genetically modified cells, to a subject.

In one embodiment, the invention provides genetically modified (i.e., edited) cells, such as a bone marrow cell, with the potential to expand or increase a population cells.

In one embodiment, the cell is a hematopoietic stem cell described herein or genetic engineered cells described herein or the progeny cells thereof are implanted with prostaglandin E2 and/or antioxidant N-acetyl-L-cysteine (NAC) to promote the engraftments of the respective cells.

In a further embodiment of any methods described herein, the hematopoietic stem cell or hematopoietic progenitor cell being contacted is of the erythroid lineage.

In one embodiment of any methods described herein, the hematopoietic stem cell or hematopoietic progenitor cell is collected from peripheral blood, cord blood, chorionic villi, amniotic fluid, placental blood, or bone marrow.

In a further embodiment of any methods described herein, the recipient subject is treated with chemotherapy and/or radiation prior to implantation of the contacted or transfected cells (i.e., the genetic engineered (i.e., edited) cells described herein).

In one embodiment, the chemotherapy and/or radiation is to reduce endogenous stem cells to facilitate engraftment of the implanted cells.

In one aspect of any method, the contacted hematopoietic stem cells described herein or genetic engineered cells described herein or the progeny cells thereof are treated ex vivo with prostaglandin E2 and/or antioxidant N-acetyl-L-cysteine (NAC) to promote subsequent engraftment in a recipient subject.

Engraftment analysis was performed 4, 8 and 12 weeks post transplantation in peripheral blood and bone marrow. For example, harvest a sample of blood from these locations and determine the BCL11A expression by any method known in the art.

In one aspect of any one method described herein, the method comprises obtaining a sample or a population of embryonic stem cells, somatic stem cells, progenitor cells, bone marrow cells, hematopoietic stem cells, or hematopoietic progenitor cells from the subject.

In one embodiment of any one method described herein, the cells that is contacted with a nucleic acid molecule describe herein, or a composition describe herein comprising a nucleic acid molecule.

In one embodiment, the somatic stem cells, progenitor cells, bone marrow cells, hematopoietic stem cells, hematopoietic progenitor cells are isolated from the host subject, transfected, cultured (optional), and transplanted back into the same host, i. e. an autologous cell transplant. In another embodiment, the embryonic stem cells, somatic stem cells, progenitor cells, bone marrow cells, hematopoietic stem cells, or hematopoietic progenitor cells are isolated from a donor who is an HLA-type match with a host (recipient) who is diagnosed with or at risk of developing SDS. Donor-recipient antigen type-matching is well known in the art. The HLA-types include HLA-A, HLA-B, HLA-C, and HLA-D. These represent the minimum number of cell surface antigen matching required for transplantation. That is the transfected cells are transplanted into a different host, i.e., allogeneic to the recipient host subject. The donor's or subject's somatic stem cells, progenitor cells, bone marrow cells, hematopoietic stem cells, or hematopoietic progenitor cells can be contacted (electroporated) with a nucleic acid molecule described herein, the contacted cells are culture expanded, and then transplanted into the host subject. In one embodiment, the transplanted cells engraft in the host subject. The transfected cells can also be cryopreserved after transfected and stored, or cryopreserved after cell expansion and stored.

In one aspect of any method, the somatic stem cell, progenitor cell, bone marrow cell, hematopoietic stem cell, or hematopoietic progenitor cell is autologous or allogeneic to the subject.

### Cells

In one embodiment, the cell is a human cell. In one embodiment, the cell is a bone marrow cell, a somatic stem cell, a progenitor cell, a hematopoietic stem cell, or a hematopoietic progenitor cell. In another embodiment of the hematopoietic cell is a cell of the erythroid lineage. Methods of isolating hematopoietic progenitor cell are well known in the art, e.g., by flow cytometric purification of CD34+ or CD133+ cells, microbeads conjugated with antibodies against CD34 or CD133, markers of hematopoietic progenitor cell. Commercial kits are also available, e.g., MACS® Technology CD34 MicroBead Kit, human, and CD34 MultiSort Kit, human, and STEMCELL™ Technology EasySep™ Mouse Hematopoietic Progenitor Cell Enrichment Kit. In one embodiment, the hematopoietic stem cells, hematopoietic progenitor cells, embryonic stem cells, somatic stem cells, or progenitor cells are collected from peripheral blood, cord blood, chorionic villi, amniotic fluid, placental blood, or bone marrow.

In one embodiment, the hematopoietic stem cells, hematopoietic progenitor cells, embryonic stem cells, somatic stem cells, or progenitor cells are collected from peripheral blood, cord blood, chorionic villi, amniotic fluid, placental blood, or bone marrow.

In one embodiment, the hematopoietic progenitor or stem cells or isolated cells can be substituted with an iPSCs described herein.

### Hematopoietic Progenitor Cell

In one embodiment, the hematopoietic progenitor cell is contacted ex vivo or in vitro. In a specific embodiment, the cell being contacted is a cell of the erythroid lineage.

“Hematopoietic progenitor cell” as the term is used herein, refers to cells of a stem cell lineage that give rise to all the blood cell types including the myeloid (monocytes and macrophages, neutrophils, basophils, eosinophils, erythrocytes, megakaryocytes/platelets, dendritic cells), and the lymphoid lineages (T-cells, B-cells, NK-cells). A “cell of the erythroid lineage” indicates that the cell being contacted is a cell that undergoes erythropoiesis such that upon final differentiation it forms an erythrocyte or red blood cell (RBC). Such cells belong to one of three lineages, erythroid, lymphoid, and myeloid, originating from bone marrow hematopoietic progenitor cells. Upon exposure to specific growth factors and other components of the hematopoietic microenvironment, hematopoietic progenitor cells can mature through a series of intermediate differentiation cellular types, all intermediates of the erythroid lineage, into RBCs. Thus, cells of the “erythroid lineage”, as the term is used herein, comprise hematopoietic progenitor cells, rubriblasts, prorubricytes, erythroblasts, metarubricytes, reticulocytes, and erythrocytes.

In some embodiment, the hematopoietic progenitor cell has at least one of the cell surface marker characteristic of hematopoietic progenitor cells: CD34+, CD59+, Thy1/CD90+, CD38lo/−, and C-kit/CD117+. Preferably, the hematopoietic progenitor cells have several of these markers.

In some embodiments, the hematopoietic progenitor cells of the erythroid lineage have the cell surface marker characteristic of the erythroid lineage: CD71 and Ter 19.

Stem cells, such as hematopoietic progenitor cells, are capable of proliferation and giving rise to more progenitor cells having the ability to generate a large number of mother cells that can in turn give rise to differentiated or differentiable daughter cells. The daughter cells themselves can be induced to proliferate and produce progeny that subsequently differentiate into one or more mature cell types, while also retaining one or more cells with parental developmental potential. The term “stem cell” refers then, to a cell with the capacity or potential, under particular circumstances, to differentiate to a more specialized or differentiated phenotype, and which retains the capacity, under certain circumstances, to proliferate without substantially differentiating. In one embodiment, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Generally, “progenitor cells” have a cellular phenotype that is more primitive (i.e., is at an earlier step along a developmental pathway or progression than is a fully differentiated cell). Often, progenitor cells also have significant or very high proliferative potential. Progenitor cells can give rise to multiple distinct differentiated cell types or to a single differentiated cell type, depending on the developmental pathway and on the environment in which the cells develop and differentiate.

In the context of cell ontogeny, the adjective “differentiated”, or “differentiating” is a relative term. A “differentiated cell” is a cell that has progressed further down the developmental pathway than the cell it is being compared with. Thus, stem cells can differentiate to lineage-restricted precursor cells (such as a hematopoietic progenitor cell), which in turn can differentiate into other types of precursor cells further down the pathway (such as an erythrocyte precursor), and then to an end-stage differentiated cell, such as an erythrocyte, which plays a characteristic role in a certain tissue type, and may or may not retain the capacity to proliferate further.

### Induced Pluripotent Stem Cells

In some embodiments, the edited cells described herein are derived from isolated pluripotent stem cells. An advantage of using iPSCs is that the cells can be derived from the same subject to which the progenitor cells are to be administered. That is, a somatic cell can be obtained from a subject, reprogrammed to an induced pluripotent stem cell, and then re-differentiated into a hematopoietic progenitor cell to be administered to the subject (e.g., autologous cells). Since the progenitors are essentially derived from an autologous source, the risk of engraftment rejection or allergic responses is reduced compared to the use of cells from another subject or group of subjects. In some embodiments, the hematopoietic progenitors are derived from non-autologous sources. In addition, the use of iPSCs negates the need for cells obtained from an embryonic source. Thus, in one embodiment, the stem cells used in the disclosed methods are not embryonic stem cells.

Although differentiation is generally irreversible under physiological contexts, several methods have been recently developed to reprogram somatic cells to induced pluripotent stem cells. Exemplary methods are known to those of skill in the art and are described briefly herein below.

As used herein, the term “reprogramming” refers to a process that alters or reverses the differentiation state of a differentiated cell (e.g., a somatic cell). Stated another way, reprogramming refers to a process of driving the differentiation of a cell backwards to a more undifferentiated or more primitive type of cell. It should be noted that placing many primary cells in culture can lead to some loss of fully differentiated characteristics. Thus, simply culturing such cells included in the term differentiated cells does not render these cells non-differentiated cells (e.g., undifferentiated cells) or pluripotent cells. The transition of a differentiated cell to pluripotency requires a reprogramming stimulus beyond the stimuli that lead to partial loss of differentiated character in culture. Reprogrammed cells also have the characteristic of the capacity of extended passaging without loss of growth potential, relative to primary cell parents, which generally have capacity for only a limited number of divisions in culture.

The cell to be reprogrammed can be either partially or terminally differentiated prior to reprogramming. In some embodiments, reprogramming encompasses complete reversion of the differentiation state of a differentiated cell (e.g., a somatic cell) to a pluripotent state or a multipotent state. In some embodiments, reprogramming encompasses complete or partial reversion of the differentiation state of a differentiated cell (e.g., a somatic cell) to an undifferentiated cell (e.g., an embryonic-like cell). Reprogramming can result in expression of particular genes by the cells, the expression of which further contributes to reprogramming. In certain embodiments described herein, reprogramming of a differentiated cell (e.g., a somatic cell) causes the differentiated cell to assume an undifferentiated state (e.g., is an undifferentiated cell). The resulting cells are referred to as “reprogrammed cells,” or “induced pluripotent stem cells (iPSCs or iPS cells).”

Reprogramming can involve alteration, e.g., reversal, of at least some of the heritable patterns of nucleic acid modification (e.g., methylation), chromatin condensation, epigenetic changes, genomic imprinting, etc., that occur during cellular differentiation. Reprogramming is distinct from simply maintaining the existing undifferentiated state of a cell that is already pluripotent or maintaining the existing less than fully differentiated state of a cell that is already a multipotent cell (e.g., a hematopoietic stem cell). Reprogramming is also distinct from promoting the self-renewal or proliferation of cells that are already pluripotent or multipotent, although the compositions and methods described herein can also be of use for such purposes, in some embodiments.

The specific approach or method used to generate pluripotent stem cells from somatic cells (broadly referred to as “reprogramming”) is not critical to the claimed invention. Thus, any method that re-programs a somatic cell to the pluripotent phenotype would be appropriate for use in the methods described herein.

Reprogramming methodologies for generating pluripotent cells using defined combinations of transcription factors have been described induced pluripotent stem cells. Yamanaka and Takahashi converted mouse somatic cells to ES cell-like cells with expanded developmental potential by the direct transduction of Oct4, Sox2, Klf4, and c-Myc (Takahashi and Yamanaka, 2006). iPSCs resemble ES cells as they restore the pluripotency-associated transcriptional circuitry and much of the epigenetic landscape. In addition, mouse iPSCs satisfy all the standard assays for pluripotency: specifically, in vitro differentiation into cell types of the three germ layers, teratoma formation, contribution to chimeras, germline transmission (Maherali and Hochedlinger, 2008), and tetraploid complementation (Woltjen et al., 2009).

Subsequent studies have shown that human iPS cells can be obtained using similar transduction methods (Lowry et al., 2008; Park et al., 2008; Takahashi et al., 2007; Yu et al., 2007b), and the transcription factor trio, OCT4, SOX2, and NANOG, has been established as the core set of transcription factors that govern pluripotency (Jaenisch and Young, 2008). The production of iPS cells can be achieved by the introduction of nucleic acid sequences encoding stem cell-associated genes into an adult, somatic cell, historically using viral vectors.

iPS cells can be generated or derived from terminally differentiated somatic cells, as well as from adult stem cells, or somatic stem cells. That is, a non-pluripotent progenitor cell can be rendered pluripotent or multipotent by reprogramming. In such instances, it may not be necessary to include as many reprogramming factors as required to reprogram a terminally differentiated cell. Further, reprogramming can be induced by the non-viral introduction of reprogramming factors, e.g., by introducing the proteins themselves, or by introducing nucleic acids that encode the reprogramming factors, or by introducing messenger RNAs that upon translation produce the reprogramming factors (see e.g., Warren et al., Cell Stem Cell, 2010 Nov. 5; 7(5):618-30). Reprogramming can be achieved by introducing a combination of nucleic acids encoding stem cell-associated genes including, for example Oct-4 (also known as Oct-3/4 or Pouf51), Sox1, Sox2, Sox3, Sox 15, Sox 18, NANOG, Klf1, Klf2, Klf4, Klf5, NR5A2, c-Myc, 1-Myc, n-Myc, Rem2, Tert, and LIN28. In one embodiment, reprogramming using the methods and compositions described herein can further comprise introducing one or more of Oct-3/4, a member of the Sox family, a member of the Klf family, and a member of the Myc family to a somatic cell. In one embodiment, the methods and compositions described herein further comprise introducing one or more of each of Oct 4, Sox2, Nanog, c-MYC and Klf4 for reprogramming. As noted above, the exact method used for reprogramming is not necessarily critical to the methods and compositions described herein. However, where cells differentiated from the reprogrammed cells are to be used in, e.g., human therapy, in one embodiment the reprogramming is not effected by a method that alters the genome. Thus, in such embodiments, reprogramming is achieved, e.g., without the use of viral or plasmid vectors.

The efficiency of reprogramming (i.e., the number of reprogrammed cells) derived from a population of starting cells can be enhanced by the addition of various small molecules as shown by Shi, Y., et al (2008) Cell-Stem Cell 2:525-528, Huangfu, D., et al (2008) Nature Biotechnology 26(7):795-797, and Marson, A., et al (2008) Cell-Stem Cell 3:132-135. Thus, an agent or combination of agents that enhance the efficiency or rate of induced pluripotent stem cell production can be used in the production of patient-specific or disease-specific iPSCs. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), vitamin C, and trichostatin (TSA), among others.

Other non-limiting examples of reprogramming enhancing agents include: Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid ((VPA) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-Cl-UCHA (e.g., 6-(3-chlorophenylureido)caproic hydroxamic acid), AOE (2-amino-8-oxo-9,10-epoxydecanoic acid), CHAP31 and CHAP 50. Other reprogramming enhancing agents include, for example, dominant negative forms of the HDACs (e.g., catalytically inactive forms), siRNA inhibitors of the HDACs, and antibodies that specifically bind to the HDACs. Such inhibitors are available, e.g., from BIOMOL International, Fukasawa, Merck Biosciences, Novartis, Gloucester Pharmaceuticals, Aton Pharma, Titan Pharmaceuticals, Schering AG, Pharmion, MethylGene, and Sigma Aldrich.

To confirm the induction of pluripotent stem cells for use with the methods described herein, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, Fbx15, Ecat1, Esg1, Eras, Gdf3, Fgf4, Cripto, Dax1, Zpf296, Slc2a3, Rex1, Utf1, and Nat1. In one embodiment, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. In some embodiments, detection does not involve only RT-PCR, but also includes detection of protein markers. Intracellular markers may be best identified via RT-PCR, while cell surface markers are readily identified, e.g., by immunocytochemistry.

The pluripotent stem cell character of isolated cells can be confirmed by tests evaluating the ability of the iPSCs to differentiate to cells of each of the three germ layers. As one example, teratoma formation in nude mice can be used to evaluate the pluripotent character of the isolated clones. The cells are introduced to nude mice and histology and/or immunohistochemistry is performed on a tumor arising from the cells. The growth of a tumor comprising cells from all three germ layers, for example, further indicates that the cells are pluripotent stem cells.

### Somatic Cells for Reprogramming

Somatic cells, as that term is used herein, refer to any cells forming the body of an organism, excluding germline cells. Every cell type in the mammalian body—apart from the sperm and ova, the cells from which they are made (gametocytes) and undifferentiated stem cells—is a differentiated somatic cell. For example, internal organs, skin, bones, blood, and connective tissue are all made up of differentiated somatic cells.

Additional somatic cell types for use with the compositions and methods described herein include: a fibroblast (e.g., a primary fibroblast), a muscle cell (e.g., a myocyte), a cumulus cell, a neural cell, a mammary cell, a hepatocyte and a pancreatic islet cell. In some embodiments, the somatic cell is a primary cell line or is the progeny of a primary or secondary cell line. In some embodiments, the somatic cell is obtained from a human sample, e.g., a hair follicle, a blood sample, a biopsy (e.g., a skin biopsy or an adipose biopsy), a swab sample (e.g., an oral swab sample), and is thus a human somatic cell.

Some non-limiting examples of differentiated somatic cells include, but are not limited to, epithelial, endothelial, neuronal, adipose, cardiac, skeletal muscle, immune cells, hepatic, splenic, lung, circulating blood cells, gastrointestinal, renal, bone marrow, and pancreatic cells. In some embodiments, a somatic cell can be a primary cell isolated from any somatic tissue including, but not limited to brain, liver, gut, stomach, intestine, fat, muscle, uterus, skin, spleen, endocrine organ, bone, etc. Further, the somatic cell can be from any mammalian species, with non-limiting examples including a murine, bovine, simian, porcine, equine, ovine, or human cell. In some embodiments, the somatic cell is a human somatic cell.

When reprogrammed cells are used for generation of hematopoietic progenitor cells to be used in the therapeutic treatment of disease, it is desirable, but not required, to use somatic cells isolated from the patient being treated. For example, somatic cells involved in diseases, and somatic cells participating in therapeutic treatment of diseases and the like can be used. In some embodiments, a method for selecting the reprogrammed cells from a heterogeneous population comprising reprogrammed cells and somatic cells they were derived or generated from can be performed by any known means. For example, a drug resistance gene or the like, such as a selectable marker gene can be used to isolate the reprogrammed cells using the selectable marker as an index.

Reprogrammed somatic cells as disclosed herein can express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; β-III-tubulin; α-smooth muscle actin (α-SMA); fibroblast growth factor 4 (Fgf4), Cripto, Dax1; zinc finger protein 296 (Zfp296); N-acetyltransferase-1 (Nat1); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fthl17; Sal14; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbxl5); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; DPPA4; other general markers for pluripotency, etc. Other markers can include Dnmt3L; Sox15; Stat3; Grb2; β-catenin, and Bmi1. Such cells can also be characterized by the down-regulation of markers characteristic of the somatic cell from which the induced pluripotent stem cell is derived.

### Pharmaceutically Acceptable Carriers

The methods of administering genetic engineered (i.e., edited) cells described herein or their progeny to a subject as described herein involve the use of therapeutic compositions comprising editedcells. Therapeutic compositions contain a physiologically tolerable carrier together with the cell composition and optionally at least one additional bioactive agent as described herein, dissolved or dispersed therein as an active ingredient. In a preferred embodiment, the therapeutic composition is not substantially immunogenic when administered to a mammal or human patient for therapeutic purposes, unless so desired.

In general, the edited cells described herein or their progeny are administered as a suspension with a pharmaceutically acceptable carrier. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation comprising cells can include e.g. osmotic buffers that permit cell membrane integrity to be maintained, and optionally, nutrients to maintain cell viability or enhance engraftment upon administration. Such formulations and suspensions are known to those of skill in the art and/or can be adapted for use with the cells as described herein using routine experimentation.

A cell composition can also be emulsified or presented as a liposome composition, provided that the emulsification procedure does not adversely affect cell viability. The cells and any other active ingredient can be mixed with excipients which are pharmaceutically acceptable and compatible with the active ingredient and in amounts suitable for use in the therapeutic methods described herein.

Additional agents included in a cell composition as described herein can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions as described herein that is effective in the treatment of a particular disorder or condition will depend on the nature of the disorder or condition, and can be determined by standard clinical techniques.

In some embodiments, the compositions of isolated edited cells described further comprises a pharmaceutically acceptable carrier. In one embodiment, the pharmaceutically acceptable carrier does not include tissue or cell culture media.

In some embodiments, the compositions of the base editor system described further comprises a pharmaceutically acceptable carrier. In one embodiment, the pharmaceutically acceptable carrier does not include tissue or cell culture media.

In some embodiments, the compositions comprising the nucleic acid molecules described further comprises a pharmaceutically acceptable carrier. In one embodiment, the pharmaceutically acceptable carrier does not include tissue or cell culture media.

### Administration & Efficacy

As used herein, the terms “administering,” “introducing” and “transplanting” are used interchangeably in the context of the placement of cells, e.g. edited cells, as described herein into a subject, by a method or route which results in at least partial localization of the introduced cells at a desired site, such as a site of injury or repair, such that a desired effect(s) is produced. The cells e.g., edited cells or their differentiated progeny can be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years, i.e., long-term engraftment. For example, in some embodiments of the aspects described herein, an effective amount of cells edited to have wild-type SBDS gene expression is administered via a systemic route of administration, such as an intraperitoneal or intravenous route.

When provided prophylactically, edited cells described herein can be administered to a subject in advance of any symptom of a SDS, as described herein. Accordingly, the prophylactic administration of the edited cells described herein serves to prevent SDS.

When provided therapeutically, edited cells described herein are provided at (or after) the onset of a symptom or indication of SDS, e.g., upon the onset of SDS.

In some embodiments of the aspects described herein, the edited cells with wild-type SBDS expression being administered according to the methods described herein comprises edited cells obtained from one or more donors. As used herein, “allogeneic” refers to a cell obtained from one or more different donors of the same species, where the genes at one or more loci are not identical. In some embodiments, syngeneic cell populations can be used, such as those obtained from genetically identical animals, or from identical twins. In other embodiments of this aspect, the edited cells are autologous cells; that is, the cells were obtained or isolated from a subject prior to editing and administered to the same subject, i.e., the donor and recipient are the same.

For use in the various aspects described herein, an effective amount of edited cells, comprises at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ hematopoietic progenitor cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The edited cells can be derived from one or more donors, or can be obtained from an autologous source. In some embodiments of the aspects described herein, the edited cells are expanded in culture prior to administration to a subject in need thereof.

In one embodiment, the term “effective amount” as used herein refers to the amount of a population of edited cells or their progeny needed to alleviate at least one or more symptom of a SDS, and relates to a sufficient amount of a composition to provide the desired effect, e.g., treat a subject having a SDS. The term “therapeutically effective amount” therefore refers to an amount of edited cells described herein or their progeny or a composition comprising edited cells described herein or their progeny that is sufficient to promote a particular effect when administered to a typical subject, such as one who has or is at risk for a SDS. An effective amount as used herein would also include an amount sufficient to prevent or delay the development of a symptom of the disease, alter the course of a symptom disease (for example but not limited to, slow the progression of a symptom of the disease), or reverse a symptom of the disease. It is understood that for any given case, an appropriate “effective amount” can be determined by one of ordinary skill in the art using routine experimentation.

As used herein, “administered” refers to the delivery of a base editor system or composition there, or cell composition as described herein into a subject by a method or route which results in at least partial localization of the cell composition at a desired site. A cell composition can be administered by any appropriate route which results in effective treatment in the subject, i.e. administration results in delivery to a desired location in the subject where at least a portion of the composition delivered, i.e. at least 1×10⁴ cells are delivered to the desired site for a period of time. Modes of administration include injection, infusion, instillation, or ingestion. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. For the delivery of cells, administration by injection or infusion is generally preferred.

In one embodiment, the cells as described herein are administered systemically. The phrases “systemic administration,” “administered systemically”, “peripheral administration” and “administered peripherally” as used herein refer to the administration of a population of edited cells other than directly into a target site, tissue, or organ, such that it enters, instead, the subject's circulatory system and, thus, is subject to metabolism and other like processes.

The efficacy of a treatment comprising a composition as described herein for the treatment of a SDS can be determined by the skilled clinician. Treatment includes any treatment of a disease in an individual or an animal (some non-limiting examples include a human, or a mammal) and includes: (1) inhibiting the disease, e.g., arresting, or slowing the progression of sepsis; or (2) relieving the disease, e.g., causing regression of symptoms; and (3) preventing or reducing the likelihood of the development of infection or sepsis.

In one embodiment, the cell to be edited contacted ex vivo or in vitro with a base editor system described herein, and the cell or its progeny is administered to the mammal (e.g., human). In one embodiment, a composition comprising an edited cell that was previously contacted with a DNA-targeting endonuclease and a pharmaceutically acceptable carrier and is administered to a mammal.

The disclosure described herein, in a preferred embodiment, does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

The disclosure described herein, in a preferred embodiment, does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Furthermore, the disclosure described herein does not concern the destruction of a human embryo.

This invention is further illustrated by the following example which should not be construed as limiting. The contents of all references cited throughout this application, as well as the figures and table are incorporated herein by reference.

### EXAMPLES

### Example 1

Approximately 90% of patients with Shwachman-Diamond-Syndrome (SDS) carry a mutation in intron 2 of the SBDS-gene (named 258+2 C to T), resulting in splicing defects and loss of protein expression. SDS patients suffer from multiple defects, but the life-threatening manifestation is bone marrow failure, i.e., the inability to produce sufficient numbers of blood cells. A high proportion of patients (30%) develops myelodysplastic syndrome and acute myeloid leukemia (AML). Currently the only curative treatment is bone marrow transplantation from a matched donor.

The homogenous mutation pattern and the fact that a point mutation causes the disease makes it a perfect target for base editing mediated correction approaches, i.e., reversing the mutated C back into the original T. While seemingly simple, this approach is complicated by multiple factors: absence of a canonical NGG-PAM site, the mutated nucleotide is framed by G's known to interfere with efficient editing, and bystander editing of a nearby C (position −4) must be avoided since this would introduce a stop codon into exon2. The advantage of base editing over alternative correction strategies is the absence of DNA double strand breaks and reduced activation of cellular defense mechanisms as compared to other gene editing approaches. It is thus considered to be particularly safe, a property of heightened importance in the context of SDS which is associated with increased genomic stability and increased risk of AML.

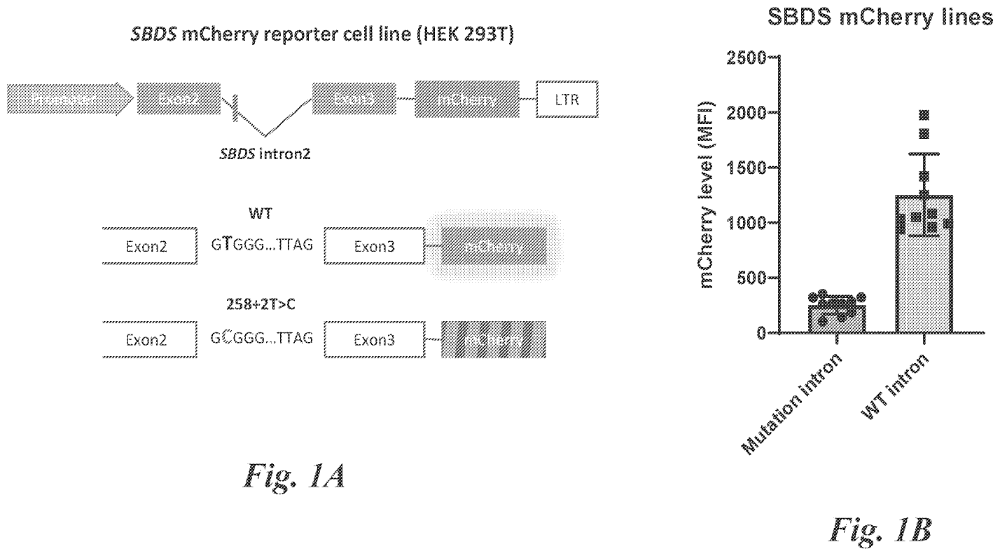

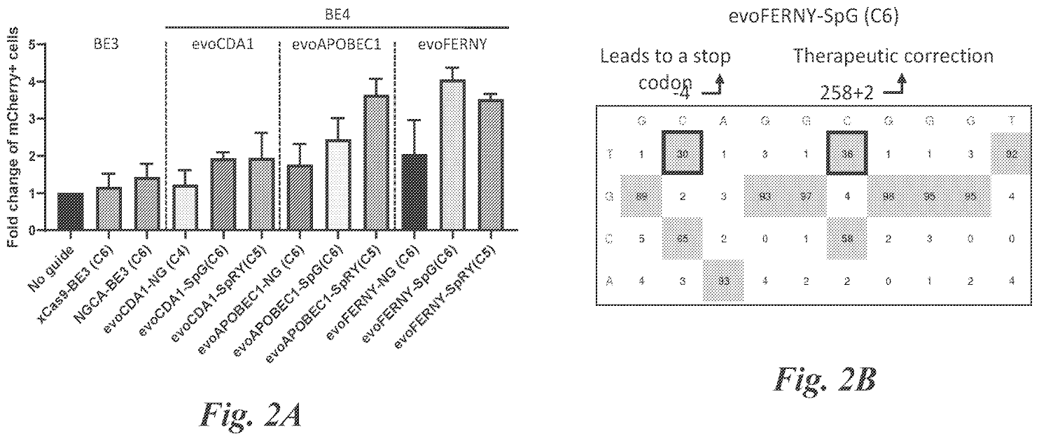

The inventors performed extensive screening of dozens of available base editing enzymes and several dozen sgRNAs in a reporter cell line generated for this purpose (FIGS. 1A, 1B, 2A, and 2B). The overwhelming majority of candidates did not lead to efficient editing, but several candidates were identified as being capable of converting the C in position 258+2 back to T with >20% efficiency (FIGS. 2A and 2B). These are: evoCDA1-SpG and evoCDA1-SpRY with sgRNAs: (C3-21 bp) GGCGGGTAACAGCTGCAGCAT (SEQ ID NO: 2), AGGCGGGTAACAGCTGC (SEQ ID NO: 3), GGCGGGTAACAGCTGCAGCA (SEQ ID NO: 4); evoAPOBEC1-SpG or evoAPOBEC1-SpRY or evoFERNY-SpG or evoRERNY-SpRY with sgRNA: (C4-17 bp) AGGCGGGTAACAGCTGC (SEQ ID NO: 5).

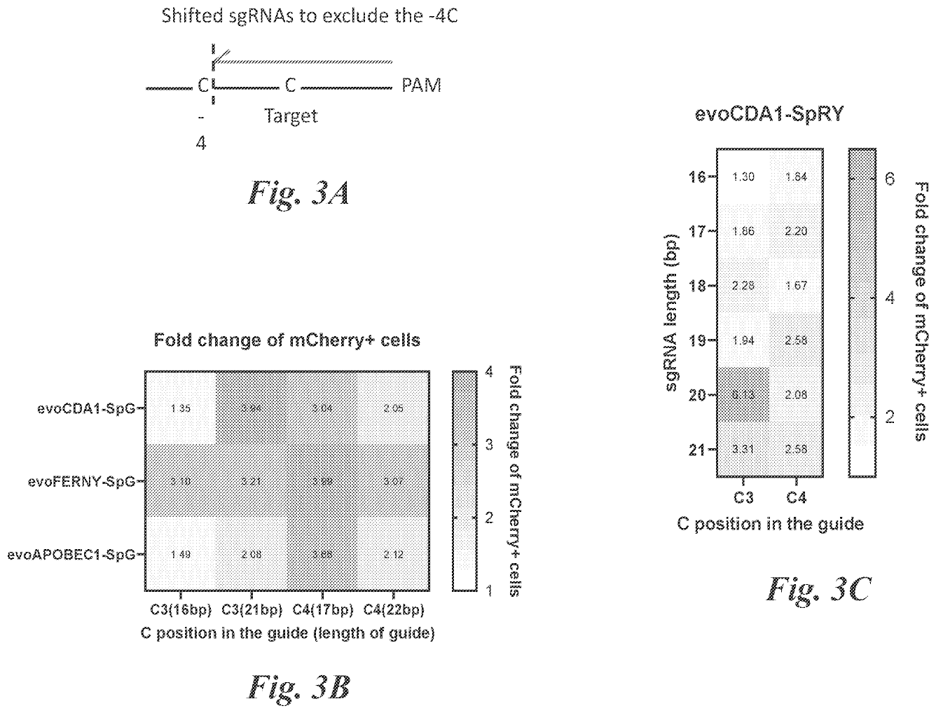

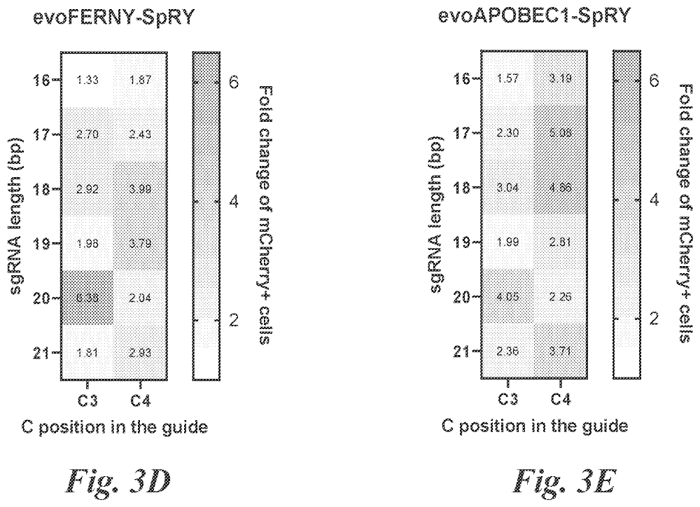

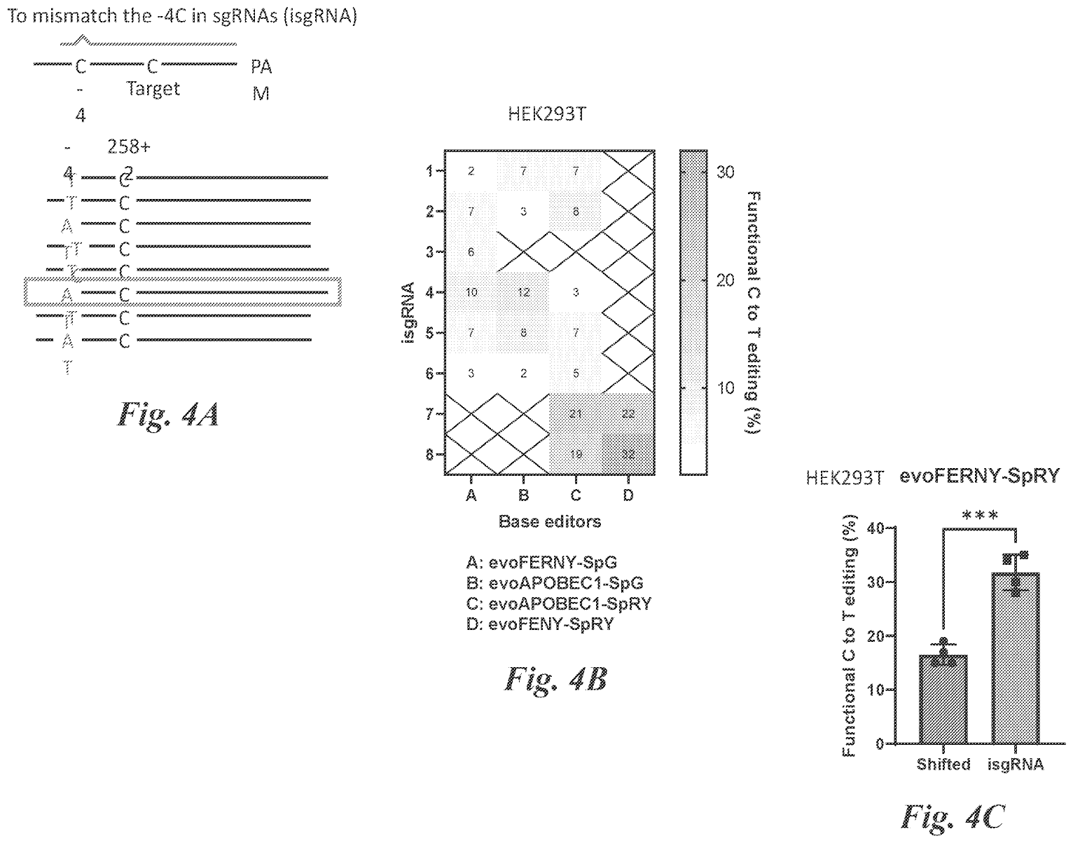

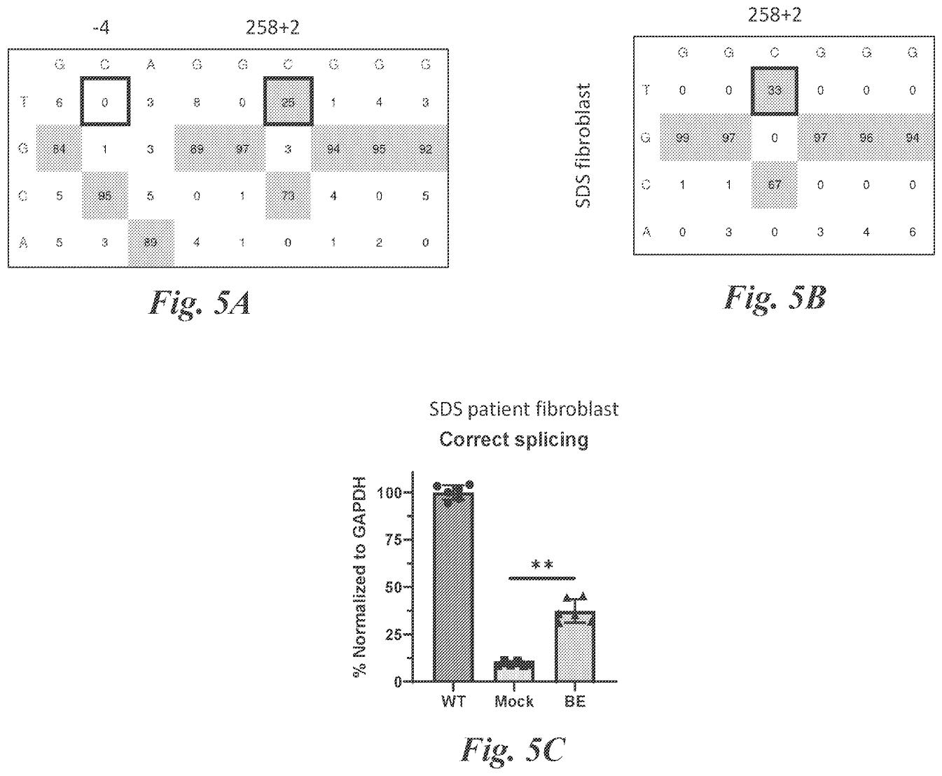

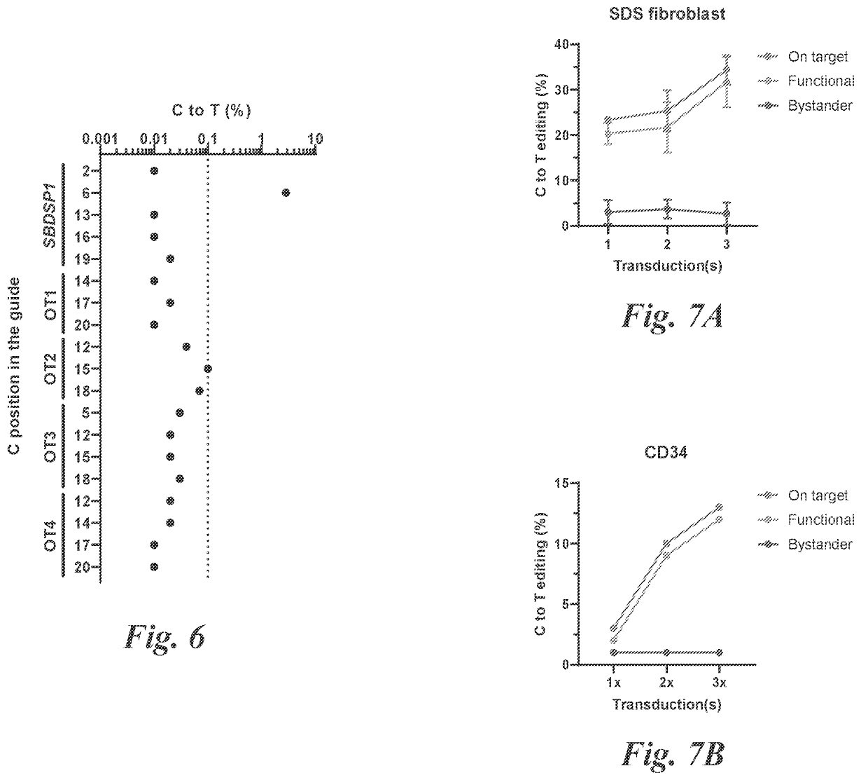

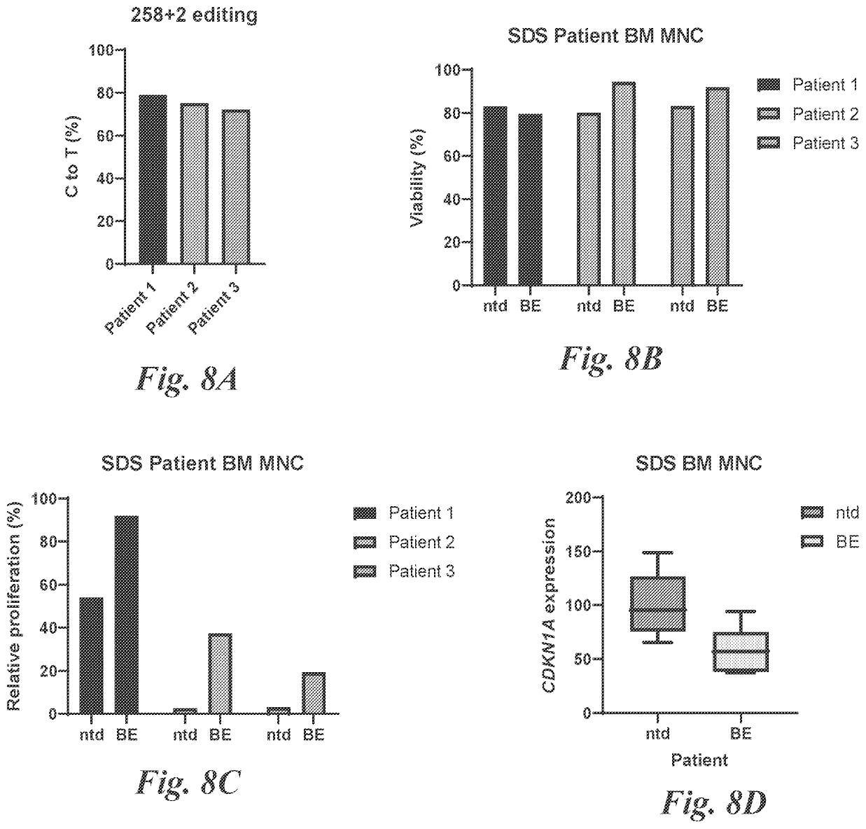

To overecome bystander editing of the nearby C in position −4 observed, additional modifications to the sgRNAs were tested. First, the sgRNAs were shifted to exclude the bystander C (FIG. 3A). Individual combinations of shifted sgRNAs with base editors led to efficient gene correction (FIGS. 3B-3E) but remained inferior to the alternative approach of using isgRNAs (FIG. 4C). isgRNAs (imperfectly pairing sgRNAs) have mismatches towards the target DNA at its 5′-end (FIG. 4A). The final optimized combination showing highest correction rates with minimal bystander editing is evoFERNY-SpRY together with the isgRNA HES980c (ATAGGCGGGTAACAGCTGCA) (SEQ ID NO: 1), where the on-target activity is maintained, and no bystander editing is detected (FIGS. 4B, 5A and 5B). Since this variant is not available as recombinant protein, mRNA electroporation into SDS patient derived fibroblasts was performed, which resulted in 33%+-8% corrected alleles, indicating monoallelic (curative) correction in −60% of the cells (FIG. 5B). This led to improvements of several SDS specific molecular and cellular phenotypes, such as recovery of correct splicing (FIG. 5C), recovery of SBDS protein expression, improvements in cell proliferation, ribosome maturation and protein synthesis (data not shown). Potential off-target editing was assessed on the top four predicted off-target sites inside or near annotated genes using the CRISPRme software. These loci were amplified by PCR and editing was assessed by amplicon sequencing, where no off target editing above the threshold was detected (FIG. 6). The editing efficiency was optimized by repeated cycles of gene editing in three SDS patient-derived fibroblast lines or healthy donor CD34+ cells (FIGS. 7A and 7B). Finally, the potency of the approach was confirmed on SDS patient bone marrow samples where between 70-80% of alleles were corrected, indicating that almost all cells were corrected. This correction was associated with improved cellular phenotypes such as higher cell viability, improved proliferation and reduced CDKN1A expression (FIGS. 8A-8D).

1. A base editor system for editing the SBDS gene comprising a base editor protein and a guide polynucleotide,
wherein the base editor protein comprises a polynucleotide programmable DNA binding domain and a cytosine deaminase domain.

2. The base editor system of claim 1, wherein the polynucleotide programmable DNA binding domain is a SpRY variant of a Streptococcus pyogenes Cas9 (SpCas9).

3. (canceled)

4. (canceled)

5. (canceled)

6. The base editor system of claim 1, wherein the cytosine deaminase domain is selected from the group consisting of evoCDA1-SpG; evoCDA1-SpRY; evoAPOBEC1-SpG; evoAPOBEC1-SpRY; evoFERNY-SpG; evoFERNY-SpRY, and evoRERNY-SpRY.

7. The base editor system of claim 1, wherein the guide polynucleotide comprises SEQ ID NO: 1, 2, 3, 4, or 5.

8. (canceled)

9. (canceled)

10. (canceled)

11. (canceled)

12. (canceled)

13. (canceled)

14. The base editor system of claim 15, wherein the SBDS gene comprises a T258C mutation.

15. The base editor system of claim 1, wherein the SBDS gene is edited at the nucleotide 258.

16. (canceled)

17. (canceled)

18. (canceled)

19. (canceled)

20. A method of editing a cell, the method comprising contacting a cell with the base editor system of claim 1.

21. The method of claim 20, wherein the cell comprises a mutation in the SBDS gene.

22. The method of claim 21, wherein the mutation in the SBDS gene is a T258C mutation.

23. The method of claim 22, wherein the base editor corrects the T285C mutation.

24. The method of claim 20, wherein the cell is selected from the group consisting of: a bone marrow cell, a somatic stem cell, a progenitor cell, a hematopoietic stem cell, or a hematopoietic progenitor cell.

25. The method of claim 24, wherein the cell is isolated prior to contacting.

26. The method of claim 23, wherein correcting the T258C mutation results in at least one of: correct splicing, increased SBDS gene product expression, increased cell proliferation, increased ribosome maturation, and increased protein synthesis in the cell.

27. The method of claim 20, wherein the cell is obtained from a subject diagnosed as having Shwachman-Diamond-Syndrome (SDS) or at risk of having Shwachman-Diamond-Syndrome (SDS).

28. The method of claim 20, wherein contacting is in vivo, in vitro, or ex vivo.

29. An edited cell produced by the method of claim 20.

30. (canceled)

31. The composition of claim 30, further comprising a pharmaceutically acceptable carrier.

32. A method of treating Shwachman-Diamond-Syndrome (SDS), the method comprising administering to the subject in need thereof a base editing system of claim 1.

33. A method of treating Shwachman-Diamond-Syndrome (SDS), the method comprising administering to the subject in need thereof an edited cell produced by the method of claim 20.

34. (canceled)

35. The method of claim 33, wherein the edited cell was obtained from the subject in need thereof prior to editing.

36. (canceled)

37. (canceled)

38. (canceled)

39. (canceled)

40. (canceled)

41. (canceled)

42. (canceled)


SEQUENCE LISTING (6 sequences)

SEQ ID NO: 1 - DNA, 20 bp, synthetic construct
  misc_feature 1..20: note = Description of sequence: Primer
  source 1..20: mol_type = other DNA
ataggcgggtaacagctgca

SEQ ID NO: 2 - DNA, 21 bp, synthetic construct
  misc_feature 1..21: note = Description of sequence: Primer
  source 1..21: mol_type = other DNA
ggcgggtaacagctgcagcat

SEQ ID NO: 3 - DNA, 17 bp, synthetic construct
  misc_feature 1..17: note = Description of sequence: Primer
  source 1..17: mol_type = other DNA
aggcgggtaacagctgc

SEQ ID NO: 4 - DNA, 20 bp, synthetic construct
  misc_feature 1..20: note = Description of sequence: Primer
  source 1..20: mol_type = other DNA
ggcgggtaacagctgcagca

SEQ ID NO: 5 - DNA, 17 bp, synthetic construct
  misc_feature 1..17: note = Description of sequence: Primer
  source 1..17: mol_type = other DNA
aggcgggtaacagctgc

SEQ ID NO: 6 - DNA, 7910 bp, Homo sapiens
  misc_feature 1..7910: note = Description of sequence: Chromosomal DNA
  source 1..7910: mol_type = genomic DNA
agacacactgtgacggctgcctgaagctagtgagtcgcggcgccgcgcactggtggttgggtcagtgccgcgcgccgatcggtcgttaccgcgaggcgctggtggccttcaggctggacggcgcgggtcagccctggttcgccggcttctgggtctttgaacagccgcgatgtcgatcttcacccccaccaaccagatccgcctaaccaatgtggccgtggtacggatgaagcgtgccgggaagcgcttcgaaatcgcctgctacaaaaacaaggtcgtcggctggcggagcggcgtgtgagtagccccctccctcgggcctgggcctgggcctgagccgtcacctccgaggcggcctgtctctgcccaagtcgagtgaatgggccaggctggggtgttggccggggaggaaatggaacattcctgctgtgagcatgagacgtcgctgtccgagcttggcgcctaagccaagggtttcttctttatttggttggttcggattgggttgttggtttggggttttgttttgttggtgtcataaaagctgcagccaagaaatctcgtaattgtggtccttttcctagaataatgatggctgagaacctagtcttacgaatactgtcatagttgtgggtacatctggtgtcatagatctcagtagccctagcagtttaaagtgcgtagtgtcttcactgctgccatctagggactggcattccgtggcagcagctgtctgcaccccaccccacccttgtgtttcttacgtcctcccatgacatttttctccagtgagcaaatggtaaggcaaatacggttctgagttttgaaaatgttccctcaggccgatgcgggcagttcacttgaggccaggagttcgaggccagcctggccaacatgaaaccccatctctactaaaaatacaaagttagccgggtgtggtggcgcatgcctgtaatcccagttactcaggaggctgaggcgggagaatcacttgaacccgggaggctgaggttacagtgacccgagatcgcgccattgcactccagcctgggcaaaaacagtgaaattccatctaggggcgggggttggggggtaagaaaaagaaaactgccctctacactaaaggtcatcagggggatttgttgtgtcttgccgttcatgttgttgccatctcgtatttaaatgtaaatgcatgtccaagtttcaagtatattcacataggactttctctcctgccctcacaagggaaaaagacctcgatgaagttctgcagacccactcagtgtttgtaaatgtttctaaaggtcaggttgccaaaaaggaagatctcatcagtgcgtttggaacagatgaccaaactgaaatctgtaagcaggtgggtaacagctgcagcatagctaaccctaataaccatttataacgtatttgtagatatattaaacattaaaggctgtttttctggaggaaagactaaccaagcaataatgtgaactgcacagtgtcacttctaataataaagaacttggtggttttgtttgttttttgtggtttttttttttttttttttgccaaaacctcctgaaatcagattttctactaaattacctcatttttctataaattgccccatttatagaatgtggtatacctgaaaactgaatttgttttcaagtatttcatctttcagatccttttttttttgggtggggggacagagtcttgctctgttgcccaggctggagtgcagtgcacttcactgcactcagctcactgcaacctctgcctcctggattcaagcgattctcttgcctcagcttccagggtagctgggattacaggtgcccaccaccctgcctggctaatttttggatttttagtagagatggggtttcaccatgttggccaggttggtctcaaactcctgaccttgggtgatatgcccgccttagccacccaaagtgctgggattacagatgtgagccactgagcccagccaaaagattcttgcatcttttgggcaaagctcaaaccattacttacatattgatagctggagaggatgaaatttaattttctctccatccagttactcattttttatggttagttaataaatagtgtgtgatagagaaagatagtgatttcttaaatgtgttggcatttttttagattttgactaaaggagaagttcaagtatcagataaagaaagacacacacaactggagcagatgtttagggacattgcaactattgtggcagacaaatgtgtgaatcctgaaacaaagagaccatacaccgtgatccttattgagagagccatgaaggacatccactattcggtgaaaaccaacaagagtacaaaacagcaggtgagtggtttctcatgtcatcaaaatatagccatggaaatcagttttctctgaagaaatcattaaaataatgggtctggggccaggcacaatggttcatgcctgtaatcctagcactttgggagccaagatgggaggattgcttgaggcctggaaacagcctgggaaacatagggacgccccatctctaaattttttttttttttttttgagacagagtcttactctattgcccaggctggagtgcagtagtatgatctcggctcactacaatctccacctcccgcgttcaagcaagtctcctgcctcagcctcctgagtagctgggattataggcacgtgccaccacactcagctaattttgtatttttagtagagttgaggtttcaccatgttggccaggctggtcttgaactcctgaccctaggtgatccgtccgccttggcctcccaaagtgctgggattacaggcatcagctaccgtaccctacctctaaattttttaatataaaaaattaaatttaaaaaaatgggtctgcatggaagcaagtgggtagaggtttggggaagagatggaggaaaaggacggggttgtaaagataggtaatttttttttttcaagtaaacacaaatatgtatataggactttcttcttcctatccaaatcaaacttttaaagacaaaacttgagtttttaaagggtcattttaacacttctttttgaattttttaatttatatataattcacataccataaatttcacactcataaagtatgtacactttaagtggtatattaacaaagttttggaaccttccctgctacctggttcgagaacattttcatcaccacaaaaagaaagtcagtatccattagtagccatcccccattttccccccacaggcccctcccaaccactaatctcctctcgttatggacttctcaattctggacatttcatataaatggaatcatacaatatgtggccttttcatggttcatacatgttgtaacctgcatcagcatgtcatttcttttttatgccggaataatagcccactgtacggaaagaaacacattttgttcattcatctatcagttgatagacattgggttgctttcacttttgagctatgatgagcaatgctgctataaaatttcttgtatgtttctgtgtagacatatgttttcatttctgtatacctggtgactaccaaacctatttctaaaacagctgcaccattttactttaccaccatcagtgtttaagagttcagtttctccacatcctcagtaatacttgtcattgtctgcctttttgatgatggccatcctggtggtatcttgtcgtggttttgatttgcatttccttaatgatgatttgagcatatttccatgtgcttattggtgcctcgtctgtcttcttttgagaaatctctgttcaggttctttgcccaccccccccgccctctttttgcaaactctgcctcccggattcaagcaattctcctgcctcagcctcttgagtagctgggattacaggcgtgcactaccacacccggctaatttttctttttttgtatttttagtggagacggggtttcaccatgttggccaggctggtctcgaattcctgaccttgtgatgcacccgcctcggcctcccaaagtgctggaattacaggcgtgagccaccacacctggccttcactttcttcatagttttttgaaacacaaaagcttttcttcttgataagtccaatttttctattttttttttaacggtcacttatgttcttaatgttatacctaagaaaccattacctaatccaactacatggaaactactttgtttttgaaaaccttatgaaataatatagtagaagaaattgcattctcgattttgtcttggtaggctttggaagtgataaagcagttaaaagagaaaatgaagatagaacgtgctcacatgaggcttcggttcatccttccagtcaatgaaggcaagaagctgaaagaaaagctcaagccactgatcaaggtcatagaaagtgaagattatggccaacagttagaaatcgtaagagtcaaatattttctttgcttcatgttacctaaatattgtattctctagtaataaatttgtagcaaacatttagatgttgtaaacgtcagatattttcttttcttttttttttttgagatggagtctcgctctgttgcccaggctggagtgcagtggtgctatctcagctcactgcaagctccgcctcccgggtacacgccattctcctgcctcagcctcctgtgtagctgggactacaggcacccgccaccaagcctggctaatttttcgtattttttagtagagacggagtttcaccgtgttagccaggacggtcttgatctcctgacctcatgatccacctgcctcagcctcccaaagtgcagggattacaggcgtgagccaccgcgcccagccagatattttcttaagactaaaggttggtcagccttgtttaccacaaatccttttatttttttcccagcatttatttttgtcatatattatgcgtagtgttttagatacatgattttagcaaatttagcaactatttaaaataagccacttaaaaattgctattttagtggaagattgataattccaattaccttaaaaattttgcaaaaattagtacaaagcacactatggaaatttacattgagttttcagagatagctcaattgcatgctttactccagtgctgatcataatcctaaaagacaatcccaaatgccacaatcccaatgttgaaatcataaaaaaatcaaaatctctaaagtccaaagcctgaacttctaatatcttgaaaatcacagttccaaaagattgaatcctggctgggcgcggtggctcatgcctgtaatcccagcactttgggaggccaaggcaggtggatcacctgaggtcgggagttcgagaccagtctgaccaacatggagaaaccctgtctctactaaaaatacaaaatagtctggcgtggtggcacatgcctgtaatcccagctactcgggaggctgaggcaggagaatcacttgaacctggaaggcagaggttgcggtgagccaagattgcaccattgcactccagcctgggcaacaagagcgaaactccatctcaaaaaaaaaaaaaaaagattgaattctgaatgatgaaattctgaaagctgaattctggcaaaaggattagcactctttgagttgtatgcaggatagtcgcgtctggttaattgcatcatgtgaggaaaagctaggaccttgttattgtcttcatttgaaaattaaaaatggtttaaagagatgagtctgggtgccaagttgataaggggtggaattgtggatgtaattttaggtgtcaacttgactggaaaggaatccctagaagcctgataaagcattgtttggggtatgtctgtgagggtgtttccagaggatattagtgtgtgaacctggtgacactaaacacagaacattttaaagaagcagcgaaaccattatgtccttatgacctcaccccacagcttcaaacacagtgtaaattgagctgtctacaaattcatgttttcaaggctttgcagcttatttatttatttatttatttatttatttttgagacagggtttcactcttattgcccaagctagagtgcaatggcgtgatctcagctcactgcaacctctgcctcccgggttcaagcgattctcctgcctcagcctcccgagtagctgggattacaggcatgtgccacctcgcccagctaattttgtatttttagtagagacggggtctctccatgttggtcaggctggtctcgaactcccgacctcaggtgatccacccgcctcggcctcccaaagtgctgggattacaggcttgagccaccgcgccctgtctgcagcttctttttaaaagtaaatgagctgtagagaagtgttgagtatgtgagtatccactgtagatgtgaactaactcatctgacactacttgaagttctaaaatctttgcaaaactgtacacatgggccaggcacagtggctcgtgcctgtaatcccagcactttgggaggccaaggtgagcagataacatggtgaaaccctatctctactaaaaatacaaaaaataagccaggtgtggtggtgggttcctgtaatcccagtttcttgggaggctgaggcaggagaatcacttgaacctgggaggcggaggctgcagtgagccaagatcacaccactgcactctatctcaaaaaaaaataaattaacatacacatggtgtctacataagtcttcacattgctttttctccttcatacgtggaggtgactttactgagctataaaatgtaatgctaaattttagtatgagaagaatcagagttttctagtttgtcccttccatttacagctgaagaatcagaataagtgtttaaacatagggattaatgccttgtcacagggggctacatggacacttgagggcagaggctaaactggaacccagtgtgccgccctacccattgtcttatctattgcaccatagaactgtggtattattagagatctggacagcattgtgcttgcctcaaaggaagttaaagctgagtttattctgtgtcttgctcatcctcatgtggtaatctgctacgttaaatgtttcaggtatgtctgattgacccgggctgcttccgagaaattgatgagctaataaaaaaggaaactaaaggcaaaggttctttggaagtactcaatctgaaagatgtagaagaaggagatgagaaatttgaatgacacccatcaatctcttcacctctaaaacactaaagtgtttccgtttccgacggcactgtttcatgtctgtggtctgccaaatacttgcttaaactatttgacattttctatctttgtgttaacagtggacacagcaaggctttcctacataagtataataatgtgggaatgatttggttttaattataaactggggtctaaatcctaaagcaaaattgaaactccaagatgcaaagtccagagtggcattttgctactctgtctcatgccttgatagctttccaaaatgaaagttacttgaggcagctcttgtgggtgaaaagttatttgtacagtagagtaagattattaggggtatgtctatacaacaaaagggggggtctttcctaaaaaagaaaacatatgatgcttcatttctacttaatggaacttgtgttctgagggtcattatggtatcgtaatgtaaagcttggatgatgttcctgattatctgagaaacagatatagaaaaattgtgccggacttacctttcattgaacatgctgccataacttagattattcttggttaaaaaataaaagtcacttatttctaattcttaaagtttataatatatattaatatagctaaaattgtatgtaatcaataaaaccactcttatgtttattaaactatggcttgtgtttctagacaa